SEQUENCE LISTING (15 sequences)

SEQ ID NO: 1 - DNA, 19 bp, Artificial
  Synthetic primer
cccaggtgatggtgtggat

SEQ ID NO: 2 - DNA, 20 bp, Artificial
  Synthetic primer
gccagcccatcataggttga

SEQ ID NO: 3 - DNA, 15 bp, Artificial
  Synthetic primer
ccatcagcccccctc

SEQ ID NO: 4 - DNA, 15 bp, Artificial
  Synthetic primer
ccatcagctcccctc

SEQ ID NO: 5 - DNA, 20 bp, Artificial
  Synthetic primer
tggccctcacttctgttctg

SEQ ID NO: 6 - DNA, 18 bp, Artificial
  Synthetic primer
ccagccggagacctacct

SEQ ID NO: 7 - DNA, 17 bp, Artificial
  Synthetic primer
aaaggtgatgtcaagcc

SEQ ID NO: 8 - DNA, 16 bp, Artificial
  Synthetic primer
aaaggtgagtcaagcc

SEQ ID NO: 9 - DNA, 22 bp, Artificial
  Synthetic primer
tgatgggagtgtcctcccgaag

SEQ ID NO: 10 - DNA, 23 bp, Artificial
  Synthetic primer
gggtaggtagtgtgtccaattac

SEQ ID NO: 11 - DNA, 21 bp, Artificial
  Synthetic primer
aggaagacttccacctccttg

SEQ ID NO: 12 - DNA, 22 bp, Artificial
  Synthetic primer
aggagtgtggtcacacagatag

SEQ ID NO: 13 - protein, 567 aa, Homo sapiens
MWLRAFILATLSASAAWGHPSSPPVVDTVHGKVLGKFVSLEGFAQPVAIFLGIPFAKPPLGPLRFTPPQPAEPWSFVKNATSYPPMCTQDPKAGQLLSELFTNRKENIPLKLSEDCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAVMVHCLRQKTEEELLETTLKMKFLSLDLQGDPRESQPLLGTVIDGMLLLKTPEELQAERNFHTVPYMVGINKQEFGWLIPMQLMSYPLSEGQLDQKTAMSLLWKSYPLVCIAKELIPEATEKYLGGTDDTVKKKDLFLDLIADVMFGVPSVIVARNHRDAGAPTYMYEFQYRPSFSSDMKPKTVIGDHGDELFSVFGAPFLKEGASEEEIRLSKMVMKFWANFARNGNPNGEGLPHWPEYNQKEGYLQIGANTQAAQKLKDKEVAFWTNLFAKKAVEKPPQTEHIEL

SEQ ID NO: 14 - protein, 567 aa, Homo sapiens
MWLRAFILATLSASAAWGHPSSPPVVDTVHGKVLGKFVSLEGFAQPVAIFLGIPFAKPPLGPLRFTPPQPAEPWSFVKNATSYPPMCTQDPKAGQLLSELFTNRKENIPLKLSEDCLYLNIYTPADLTKKNRLPVMVWIHGGELMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAVMVHCLRQKTEEELLETTLKMKFLSLDLQGDPRESQPLLGTVIDGMLLLKTPEELQAERNFHTVPYMVGINKQEFGWLIPMQLMSYPLSEGQLDQKTAMSLLWKSYPLVCIAKELIPEATEKYLGGTDDTVKKKDLFLDLIADVMFGVPSVIVARNHRDAGAPTYMYEFQYRPSFSSDMKPKTVIGDHGDELFSVFGAPFLKEGASEEEIRLSKMVMKFWANFARNGNPNGEGLPHWPEYNQKEGTLQIGANTQAAQKLKDKEVAFWTNLFAKKAVEKPPQTEHIEL

SEQ ID NO: 15 - protein, 298 aa, Homo sapiens
MWLRAFILATLSASAAWGHPSSPPVVDTVHGKVLGKFVSLEGFAQPVAIFLGIPFAKPPLGPLRFTPPQPAEPWSFVKNATSYPPMCTQDPKAGQLLSELFTNRKENIPLKLSEDCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGESSPWLSKLLSLLGAKPPPLLSWFTACDRRRKRSSWRRH

(19) United States
(12) Patent Application Publication
(10) Pub. No.: US 2011/0020801 A1
(43) Pub. Date: Jan. 27, 2011

(54) Carboxylesterase-1 Polymorphisms and Methods of Use Therefor

(76) Inventors: John S. Markowitz, Charleston, SC (US); Haojie Zhu, Charleston, SC (US)

Correspondence Address: FULBRIGHT & JAWORSKI L.L.P., 600 CONGRESS AVE., SUITE 2400, AUSTIN, TX, 78701, US

(21) Appl. No.: 12/663,644

(22) PCT Filed: Jun. 9, 2008
(86) PCT No.: PCT/US08/66280

§ 371 Date: Oct. 4, 2010

Related U.S. Application Data
(60) Provisional application No. 61/051,680, filed on May 9, 2008.
(60) Provisional application No. 60/942,818, filed on Jun. 8, 2007.
(60) Provisional application No. 61/053,524, filed on May 15, 2008.

(51) Int. Cl. C12Q 1/68; G01N 33/53

(52) U.S. Cl. 435/6; 435/7.2

(57) ABSTRACT

Methods and kits are provided for detecting polymorphisms in carboxylesterase-1 (CES1). Several single nucleotide polymorphisms (SNPs) in CES1 in humans, and methods for detecting the same, are provided (e.g., Gly143Glu, 12754T>del). Results indicate that the Gly143Glu (9486G>A) polymorphism has an allelic frequency of 1.5% in the Caucasian population. Polymorphisms of the present invention may alter the function of the carboxylesterase-1 enzyme (hCES1). Thus, the methods and kits of the present invention may be used to personalize a therapy and/or avoid adverse consequences of altered metabolism of a therapeutic or compound (e.g., enalapril, methylphenidate, etc.) which may result due to a CES1 polymorphism. In addition, recombinant cells lines overexpressing wild-type CES1 or expressing CES1 mutants are provided. Such cell lines may be used to assess the effects of candidate compounds on CES1, and the action of CES1 on these candidate compounds.

### RELATED APPLICATIONS

This application claims priority to U.S. Provisional Application Ser. Nos. 60/942,818 filed Jun. 8, 2007; 61/051,680 filed May 9, 2008; and 61/053,524 filed May 15, 2008, the entire contents of which are hereby incorporated by reference.

### GOVERNMENT INTEREST CLAUSE

This invention was made with government support under grant nos. RO1 DA-15797 and M01 RR01070-18 awarded by the National Institutes of Health. The government has certain rights in the invention.

### BACKGROUND OF THE INVENTION

1. Field of the Invention

The present invention relates generally to the fields of molecular biology and medicine. More particularly, it concerns methods and kits for detecting polymorphisms in the carboxylesterase-1 (hCE-1) enzyme.

2. Description of Related Art

The carboxylesterase-1 (CES1) gene encodes for human carboxylesterase-1 (hCES1), the principal enzyme governing the metabolism of methylphenidate (MPH) and numerous other conventional and illicit drugs including heroin and cocaine.^{1,2} Single nucleotide polymorphisms (SNPs) can significantly impact the expression and/or activity of drug metabolizing enzymes and transporters and thus contribute to interindividual variability in pharmacokinetics and therapeutic response.

Methylphenidate (Ritalin®) is an example of a drug³ which is metabolized by hCES1. Methylphenidate is the most common pharmacologic agent used to treat attention-deficit hyperactivity disorder which afflicts school-age children with an estimated worldwide prevalence of 8-12%.^{3,4} Significant interindividual variability in MPH pharmacokinetics and pharmacodynamics is well recognized yet remains unexplained. The primary metabolic pathway governing the metabolism of MPH is rapid deesterification to the inactive metabolite, ritalinic acid (FIG. 1).⁷ This process is mediated by hCES1.⁸

Significant adverse effects can result as a consequence of altered metabolism or pharmacokinetics of a drug between individuals. Adverse effects (e.g., toxicity, etc.) may occur when a “typical” amount of a therapeutic, e.g., methylphenidate (Ritalin®) or enalapril (Vasotec®), is administered to an individual and results in an “atypical” concentration (e.g., blood concentration) due to altered metabolism of the therapeutic. For example, therapeutic doses of methylphenidate can occasionally cause significant increases in a number of cardiovascular parameters due to both central dopaminergic effects and increased plasma epinephrine concentrations.¹⁴⁻¹⁶ In rare instances, stroke or sudden death have been reported in patients with underlying risk factors and has led to the United States Food and Drug Administration to mandate that drug manufacturers of psychostimulants provide educational literature on the risks to patients.¹⁷

The probability of avoiding these adverse consequences would be greatly improved if methods were available to predict altered metabolism of a compound or therapeutic in a subject prior to determining whether or not to administer the compound or therapeutic to the subject. Clearly, there exists a need for improved methods for detecting the underlying causes that result in altered metabolism or pharmacodynamics of therapeutics. In addition, the identification of molecules that modulate such pharmacodynamics would have considerable benefit.

### SUMMARY OF THE INVENTION

The present invention overcomes limitations in the prior art by providing methods for the detection of specific polymorphisms in the carboxylesterase-1 (CES1) gene which may alter hCES1 function. These polymorphisms in the CES1 gene (Gly143Glu, 12754T>del) are present in the human population. The data provided herein indicates that either or both of these mutations can lead to clinically significant alterations in pharmacokinetics in response to methylphenidate and other hCES1 substrates. The polymorphisms of the present invention may underlie certain interindividual variation in responses to hCES1 substrates. The allelic frequency of the Gly143Glu polymorphism has been calculated to be about 1.5% in the Caucasian population.

An aspect of the present invention relates to a method of diagnosing reduced carboxylesterase-1 function in a subject, wherein the method comprises detecting the presence or absence of at least one of 12754T>del or Gly143Glu (9486G>A) in the carboxylesterase-1 gene in a biological sample from the subject, wherein the presence of at least one of 12754T>del or Gly143Glu (9486G>A) indicates that the subject has reduced carboxylesterase-1 function. The method may comprise detecting the presence or absence of one or both of 12754T>del and/or Gly143Glu (9486G>A) in the carboxylesterase-1 gene. In certain embodiments, the detecting comprises real-time PCR (rtPCR). The rtPCR may utilize a first oligonucleotide probe comprising a 5′ fluorescent dye and a second oligonucleotide probe comprising a 3′ quencher. The rtPCR may utilize a 5′ nuclease probe, a TaqMan° probe, a molecular beacon, or a FRET probe.

In certain embodiments, the rtPCR utilizes: 5′-CCCAGGTGATGGTGTGGAT-3′ (SEQ ID NO:1), 5′-GCCAGCCCATCATAGGTTGA-3′ (SEQ ID NO:2), 5′-CCATCAGCCCCCCTC-3′ (SEQ ID NO:3), and 5′-CCATCAGCTCCCCTC-3′ (SEQ ID NO:4). SEQ ID NO:3 may be Vic labeled and SEQ ID NO:4 may be Fam labeled.

In other embodiments, the rtPCR utilizes: 5′-TGGCCCTCACTTCTGTTCTG-3′ (SEQ ID NO:5), 5′-CCAGCCGGAGACCTACCT-3′ (SEQ ID NO:6), 5′-AAAGGTGATGTCAAGCC-3′ (SEQ ID NO:7), and 5′-AAAGGTGAGTCAAGCC-3′ (SEQ ID NO:8). SEQ ID NO:7 may be Vic labeled and SEQ ID NO:8 may be Fam labeled.

The subject may be a human. The biological sample may be blood, sputum, saliva, mucosal scraping, or a tissue biopsy. In certain embodiments, the method is further defined as a method of individualizing a therapy for the subject. Said individualizing may comprise determining an appropriate amount of methylphenidate to administer to the subject. The individualizing may comprise determining an appropriate dose of a drug to be administered to the subject, wherein the drug is an opioid, meperidine, a dopaminergic or noradrenergic drug, methylphenidate, an ACE inhibitor, quinapril, enalapril, benzapril, imidapril, delapril, pemocapril, cilazapril, an anesthetic, lidocaine, lovastatin, an antiviral drug, oseltamivir, an anti-cancer drug, or irinotecan.

In certain embodiments, the method is further defined as a method of determining the sensitivity of the subject to a compound. The compound may be selected from the group consisting of heroin, cocaine, a toxin, a chemical warfare agent, sarin nerve gas, soman, tabun, an insecticide, and an organophosphate insecticide. DNA and/or RNA may be isolated from the sample. A nucleic acid probe may be hybridized to DNA obtained or derived from the sample. The probe may be detectably labeled. The probe may be from 15 to 25 nucleotides long. The probe may be single-stranded or double-stranded. The label is a radioisotope, a bioluminescent compound, a chemiluminescent compound, a fluorescent compound, a metal chelate, or an enzyme. The fluorescent compound may be a Vic label, a Fam label, a TaqMan° label, fluorescein, rhodamine, auramine, Texas Red, AMCA blue, or Lucifer yellow. In certain embodiments, at least part of the carboxylesterase-1 gene of the subject is amplified prior to detection. The amplifying may be via polymerase chain reaction (PCR).

The subject may be heterozygous or homozygous for at least one of said 12754T>del or Gly143Glu (9486G>A). In certain embodiments, the subject has neither said 12754T>del or Gly143Glu (9486G>A). The detecting may comprise sequencing at least part of the carboxylesterase-1 gene of the subject.

Another aspect of the present invention relates to kit for detecting the presence or absence of Gly143Glu (9486G>A) in a carboxylesterase-1 gene comprising a nucleic acid probe in a suitable container means, wherein the nucleic acid probe can selectively bind the 9486 nucleotide of the carboxylesterase-1 gene. In certain embodiments, the kit comprises: 5′-CCCAGGTGATGGTGTGGAT-3′ (SEQ ID NO:1), 5′-GCCAGCCCATCATAGGTTGA-3′ (SEQ ID NO:2), 5′-CCATCAGCCCCCCTC-3′ (SEQ ID NO:3), and 5′-CCATCAGCTCCCCTC-3′ (SEQ ID NO:4). In certain embodiments, SEQ ID NO:3 is Vic labeled and SEQ ID NO:4 is Fam labeled. The kit may comprise real-time PCR reagents. The kit may further comprise a test for a CYP2D6 polymorphism.

Yet another aspect of the present invention relates to a kit for detecting the presence or absence of 12754T>del in a carboxylesterase-1 gene comprising a nucleic acid probe in a suitable container means, wherein the nucleic acid probe can selectively bind the 12754 nucleotide of the carboxylesterase-1 gene. In certain embodiments, the kit comprises: 5′-TGGCCCTCACTTCTGTTCTG-3′ (SEQ ID NO:5), 5′-CCAGCCGGAGACCTACCT-3′ (SEQ ID NO:6), 5′-AAAGGTGATGTCAAGCC-3′ (SEQ ID NO:7), and 5′-AAAGGTGAGTCAAGCC-3′ (SEQ ID NO:8). In certain embodiments, SEQ ID NO:7 is Vic labeled and SEQ ID NO:8 is Fam labeled. The kit may comprise real-time PCR reagents. The kit may further comprise a test for a CYP2D6 polymorphism.

Embodiments of the technology entail a cell line specifically over expressing the gene CES1 encoding for the major hydrolytic enzyme known as carboxylesterase-1 (hCES1) as well as naturally occurring genetic variant which occurs in a significant percentage of the general population. hCES1 is an endogenous enzyme found predominately in the liver which can both deactivate certain compounds (e.g., methylphenidate [Ritalin®]) or activate a variety of medications formulated as so called prodrugs (e.g., oseltamivir [Tamiflu®]) which are dependent on functional enzyme to convert the administered parent drug (prodrug) to its therapeutically active moiety.

Applications for the subject cell lines developed via transfection and the site-directed mutagenesis have a number of high throughput in vitro applications including: (1) the ability to study and assess existing therapeutic agents and/or lead compounds as potential hCES1 substrates and/or inhibitors; (2) the ability to assess the relative catalytic efficiency of the wildtype (i.e., normal) enzyme versus mutated enzyme can be rapidly tested with this system; (3) since hCES1 is stereoselective relative to specific substrates, this too can be assessed for racemic compounds; and (4) an assessment of the potential influence of the genetic variant in those individuals carrying this mutation can be made to assess their ability to metabolize (i.e., deactivate versus activate) a variety of compounds.

Embodiments of the technology can be used as a rapid screening tool for assessing whether (1) existing therapeutic agents or lead compounds are substrates of hCES1 or inhibitors of this enzyme with implications for drug-drug interaction potential; and (2) to screen existing therapeutic agents or lead compounds (during drug discovery and development process) in determining the influence of specific genetic mutations on drug disposition and/or therapeutic action or toxicity.

In particular, the present invention also provides for an isolated cell that overexpresses wild-type carboxylesterase-1 (CES1) relative to expression of CES1 in a normal cell. Another embodiment comprises an isolated cell that contains an heterologous expression construct encoding mutant carboxylesterase-1 (CES1) under the control of a promoter operable in said cell. The mutant CES1 may comprise a Gly143→Glu substitution or a T deletion at genomic nucleotide 12754. The promoter may be a native CES1 promoter. The cell may be an embryonic kidney cell.

In another embodiment, there is provided a method of assessing the effect of carboxylesterase-1 (CES1) activity on a candidate substance comprising (a) providing a cell that expresses mutant CES1 or overexpresses, relative to a normal cell, wild-type CES1; (b) contacting said cell with said candidate substance; and (c) assessing the effect of CES1 on said candidate substance. Assessing may comprise measuring modification of said candidate substance by CES1. Assessing may also comprise measuring levels of said candidate substance in said cell and/or in media in which said cell is cultured. Modification may also comprise conversion of a prodrug candidate substance to an active moiety, or isomerization of said candidate substance. Measuring may comprise chromatography or mass spectrometry. The mutant CES1 may comprise a Gly143→Glu substitution or a T deletion at genomic nucleotide 12754.

In another embodiment, there is provided a method of assessing the effect of a candidate substance on carboxylesterase-1 (CES1) activity comprising (a) providing a cell that expresses mutant CES1 or overexpresses, relative to a normal cell, wild-type CES1; (b) contacting said cell with said candidate substance; and (c) assessing the effect of said candidate substance on CES1 expression or activity. Assessing may comprise measuring candidate substance modification by CES1. The method may further comprise contacting said cell with a known substrate of CES1, and wherein assessing comprises measuring modification of said known substrate. The mutant CES1 may comprise a Gly143→Glu substitution or a T deletion at genomic nucleotide 12754.

In still a further embodiment, there is provided a method of assessing the effect of a candidate substance on a cell having a carboxylesterase-1 (CES1) deficiency comprising (a) providing a cell that expresses mutant CES1; (b) contacting said cell with said candidate substance; and (c) assessing the effect of said candidate substance on said cell. Assessing may comprise measuring CES1 activity or expression, or modification of a CES1 substrate, such as conversion of a prodrug substrate to an active moiety, or isomerization of said substrate. Assessing may also comprise measuring levels of said candidate substance in said cell and/or in media in which said cell is cultured. The mutant CES1 may comprise a Gly143→Glu substitution or a T deletion at 12754.

In still yet another embodiment, there is provided a method of assessing the effect of a carboxylesterase-1 (CES1) mutation on CES1 activity comprising (a) providing a cell that expresses mutant CES1; (b) contacting said cell with a CES1 substrate; and (c) assessing modification of said substrate by said CES1.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: The metabolic pathway of racemic MPH in humans.

FIG. 2: The plasma concentration versus time curve of total (d-MPH and l-MPH) concentrations of MPH in an apparent aberrant metabolizer compared to a similar plot of mean isomer concentrations from 19 study peers following a single 0.3 mg/kg dose of racemic MPH.

FIG. 3: The plasma concentration versus time curve presenting individual isomer concentrations (d- vs l-MPH) in a slow metabolizer compared to an AUC representing the mean values of 19 study peers following a single 0.3 mg/kg dose of racemic MPH.

FIG. 4: Alignment of the predicted protein sequences of wildtype hCES1 (SEQ ID NO:13) and the two mutations identified in the slow-metabolizer. p.Gly143Glu (SEQ ID NO:14) is the Gly143Glu substitution and is denoted by the boxed amino acid. p.Asp260fs (SEQ ID NO:15) is the Asp260Glu frameshift mutation and the altered amino acid sequence is underlined. The amino acids of the catalytic triad are bolded, the residues of the oxyanion hole are indicated by the symbol ‘ø’, and * designates a stop codon.

FIG. 5: The interaction of hCES1 with d- and l-MPH (from Sun et al., JPET 2004). Note position of Gly143 relative to d- and l-MPH.

FIG. 6: Catalytic activity of WT CES1 and its variants on d-MPH and l-MPH hydrolysis. After d- and l-MPH (20 μM-1000 μM) were incubated with S9 (500 μg/ml) from cells transfected with WT, p.Gly143Glu, and p.Asp260fs CES1 at 37° C. for 2 h, the produced RA was measured by HPLC assay. Significant catalytic stereoselectivity of WT CES1 was found in catalyzing d- and l-MPH hydrolysis. p.Gly143Glu and p.Asp260fs failed to show any hydrolytic activity on both d- and l-isomer of MPH. Data are means±S.D. for three independent experiments.

FIG. 7: Hydrolysis of PNPA by CES1 and its mutants. Cell S9 fractions prepared from cells transfected with cDNA constructs encoding WT, p.Gly143Glu, and p.Asp260fs CES1 were assayed for their catalytic activity to PNPA hydrolysis. The hydrolytic product PNP was monitored by the absorbance at 405 nm after incubating PNPA with cell S9 (20 μg/ml) at 37° C. for 10 min. Data were expressed as the mean±S.D. (n=3).

FIG. 8: Enzymatic activity of WT and mutant hCES1 on oseltamivir hydrolysis. The hydrolytic activity of WT hCES1 and its variants on oseltamivir were evaluated by measuring the active metabolite oseltamivir acid utilizing an established HPLC assay. Profoundly decreased enzymatic activity was observed in p.Gly143Glu compared to WT enzyme. No catalytic activity was found in p.Asp260fs variant. Data were expressed as the mean±S.D. for four independent experiments.

FIGS. 9A-B: (FIG. 9A) Western blotting analysis of hCES1 expression in the Flp-In-293 cells transfected with or without hCES1 cDNA, comparing to hCES1 expression in human liver cells. Anti-action was included as the sample loading control. (FIG. 9B) Enzymatic activity of hCES1 on PNPA hydrolysis. The catalytic activity was determined by measuring the hydrolytic product PNP with s9 prepared from human liver cells and hCES1 transfected Flp-In-293 cells.

FIG. 10: Western blotting analysis of hCES1 expression in the cells transfected with V5-His tagged and untagged hCES1 cDNA. Anti-action was included as the sample loading control.

FIG. 11: Enzymatic activity of V5-His tagged and untagged hCES1 on PNPA hydrolysis. The catalytic activity was determined by measuring the hydrolytic product PNP after incubating 100 μM PNPA with s9 (20 μg/ml) prepared from tagged and untagged hCES1 transfected cells at 37° C. for 10 min. Data were expressed as the mean±S.D. (n=4).

FIG. 12: Activation of oseltamivir mediated by hCES1 in humans.

FIG. 13: The chromatogram of RA HPLC analysis after 100 μM of l-MPH incubated with WT hCES1 s9 (0.5 mg/ml) at 37° C. for 2 hours.

FIG. 14: The typical chromatogram of 100 μM oseltamivir phosphate hydrolyzed by WT hCES1 s9 (0.1 mg/ml) at 37° C. for 10 min.

### DESCRIPTION OF ILLUSTRATIVE EMBODIMENTS

The present invention provides single nucleotide polymorphisms (SNP) which may be used to diagnose altered human carboxylesterase-1 (hCES1) function. Two polymorphisms in the human carboxylesterase-1 gene (CES1) are provided. One SNP is a mutation in exon 4 is located in codon 143 (GGG→GAG) which leads to the nonconservative glycine 143 to glutamic acid amino acid substitution (Gly143Glu, p.Gly143Glu). The other SNP is a deletion in exon 6 at codon 260 which results in a frameshift mutation that alters residues 260-299 and causes early truncation at a premature stop codon (12754T>del, p.Asp260fs). The allelic frequency of the Gly143Glu has been calculated to be ˜1.5% in the Caucasian population.

These polymorphisms can affect carboxylesterase-1 function. For example, either or both of these mutations may result in altered methylphenidate (MPH) pharmacokinetics. These polymorphisms led to grossly elevated MPH blood concentrations along with unprecedented concentrations of the l-MPH isomer in an otherwise normal volunteer heterozygous for both SNPs on separate alleles of the CES1 gene. This volunteer is referred to herein as the “slow metabolizer.”

The data presented herein indicates that polymorphisms of the present invention may underlie some of the inter-individual variation in responses to CES-1 substrates (e.g., metabolism of methylphenidate). In order to determine if the variations in hCES1 activity led to a quantifiable difference in the pharmacodynamic effects of MPH, the hemodynamic response of the slow metabolizer was compared to his study peers that received 0.3 mg/kg MPH. Although the slow metabolizer did not report adverse effects to the study staff, the subject achieved statistical outlier status for the systolic blood pressure (SBP), diastolic blood pressure (DBP), and heart rate (HR), the mean arterial pressure (MAP) endpoints using data obtained 1.5 hours after MPH administration. Therapeutic doses of MPH can occasionally cause significant increases in a number of cardiovascular parameters due to both central dopaminergic effects and increased plasma epinephrine concentrations.¹⁴⁻¹⁶ In rare instances stroke or sudden death was reported in patients with underlying risk factors and has led to the United States Food and Drug Administration to mandate that drug manufacturers of psychostimulants provide educational literature on the risks to patients.¹⁷ The results indicate a possible increased risk of adverse events in individuals with the identified CES1 polymorphisms, a risk which could be increased after multiple dosing or the use of one daily modified release formulations which presently dominate the market.^{5,9} Thus, the present invention permits the individualization of therapies to avoid such adverse events.

### I. CARBOXYLESTERASE-1 AND SUBSTRATES THEREOF

The carboxylesterase 1 (CES1) gene (MIM 114835) encodes for human carboxylesterase 1 (CES1), the principal enzyme governing the metabolism of the most widely prescribed psychostimulant methylphenidate (MPH) and is involved in the metabolism of numerous other therapeutic medications as well as some illicit drugs such as heroin and cocaine. Further, it is responsible for the metabolic activation of a number of ester prodrugs. Single nucleotide polymorphisms (SNPs) can significantly influence the metabolism and disposition of many therapeutic agents.

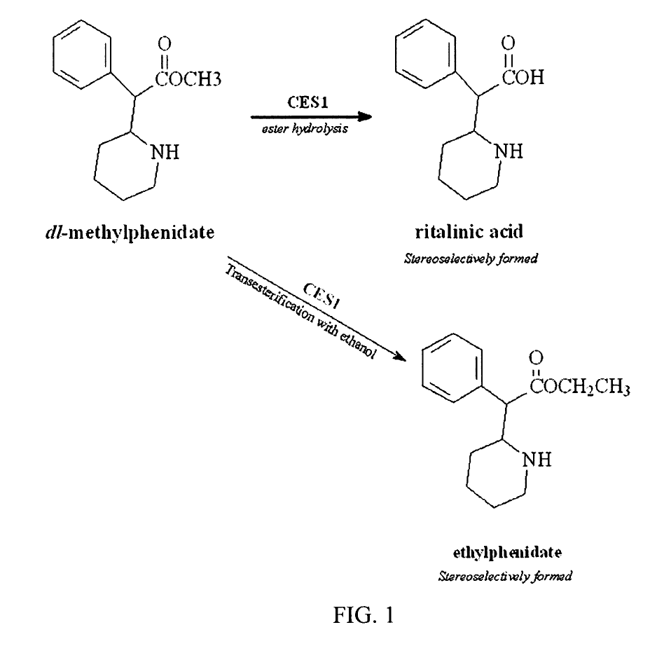

Striking interindividual differences exist between d- and l-MPH metabolism and disposition and have consistently been demonstrated in enantiospecific investigations. The major pathway mediating the metabolism of MPH is rapid deesterification to the inactive metabolite ritalinic acid. The most common formulation of MPH is the racemic mixture of d-threo-(R,R)- and l-threo-(S,S)-MPH with the d-isomer regarded as the active isomer. The primary metabolic pathway governing the metabolism of both d- and l-MPH is deesterification to the inactive metabolite, ritalinic acid (FIG. 1). This process, mediated by CES1, is stereoselective and heavily favors the hydrolysis of the l-isomer. Indeed, pharmacokinetic studies of racemic MPH which have employed enantioselective analytical methods have consistently demonstrated that the l-isomer accounts for only a small fraction (1-15%) of the total circulating blood concentrations of MPH with the predominant circulating species being the d-MPH isomer.^{5,9} Furthermore, the plasma half-life (t_{1/2}) of d-MPH is markedly longer than that of l-MPH. The pre-systemic metabolism and clearance of dl-MPH is an enantioselective process resulting in markedly higher plasma concentrations of d-MPH relative to l-MPH. In an enantiospecific study of MPH administered intravenously, both isomers exhibited similar distribution characteristics, though the terminal elimination phase of the l-isomer was more rapid. In a number of other studies utilizing various oral formulations, the area under the plasma concentration versus time curve (AUC_{inf}) value for the 1-isomer has been reported to only reach approximately 1%-15% of that of d-MPH.²¹

Prodrugs. Prodrugs are typically defined as any compound that must undergo biotransformation. before exhibiting its intended pharmacological effects. Therefore prodrugs can be viewed as compounds which have incorporated specialized non-toxic protective groups which are intended to exist only transiently to alter or eliminate undesirable characteristics of the active compound. Such undesirable qualities or impediments to adequate delivery of the therapeutic moiety to the intended site of action often relate to poor aqueous solubility, absorption and permeability as well as high first-pass hepatic extraction-all factors which contribute to overall poor oral bioavailability.

Thus, the common rationale and strategy behind the synthesis of prodrugs is to increase lipohilicity and mask hydrogen bonding groups of an active compound through the addition of another moiety-most commonly an ester. Indeed, the design of a number of prodrugs exploit the endogenous hydrolase activity known to occur in several tissues as the basis for the synthesis of a number of medications containing ester linkages.¹¹ Such prodrugs must be stable to hydrolytic processes during the absorption phase yet readily undergo enzymatic hydrolysis liberating the active moiety upon reaching the systemic circulation and passing through the liver.²⁵

Although carboxylesterases (CESs) are found within the blood of essentially all mammals studied including those routinely used in laboratory research such as rodents, it is of interest and of importance to note that there is reportedly no significant CES mediated hydrolytic activity detected in human plasma.²⁶ However, a full assessment of the prodrug's toxicology is often left to the sponsor's own initiative during the NDA process, and in many cases the active (i.e., liberated) drug's toxicity profile is claimed by the sponsor to represent the toxicity profile produced when the prodrug alone is administered.²⁷

Clearly, this approach can neglect the adequate assessment of the parent (i.e., prodrug's) toxicity. By analogy, the terfenadine (Seldane®) experience in which metabolic inhibition of the activating enzyme (CYP3A4) of this prodrug by a coadministered medication resulted in unexpectedly high concentrations of the prodrug and ensuing cardiotoxicity and a number of deaths. However, with regard to assessing the potential effects of a genetically deficient individual relative to human CES1 (hCES1) activity, there are available compounds which are known to inhibit carboxylesterase activity (e.g., loperamide, benzil) but none are specific for hCES1 only or have further limitations regarding the safe administration to human subjects.

### II. POLYMORPHISMS OF THE PRESENT INVENTION AFFECT hCES1 FUNCTION

Either or both of the polymorphisms identified herein may result in decreased functional activity of hCES1. The hCES1 enzyme belongs to a larger family of serine hydrolases, which include human acetylcholinesterase (AcChE) and butyrylcholine esterase (BuChE). Crystal structures of human CES1, AcChE, and BuChE indicate that each has an analogous active site groove containing a catalytic triad consisting of a serine, a glutamic acid, and a histidine residue.¹⁹

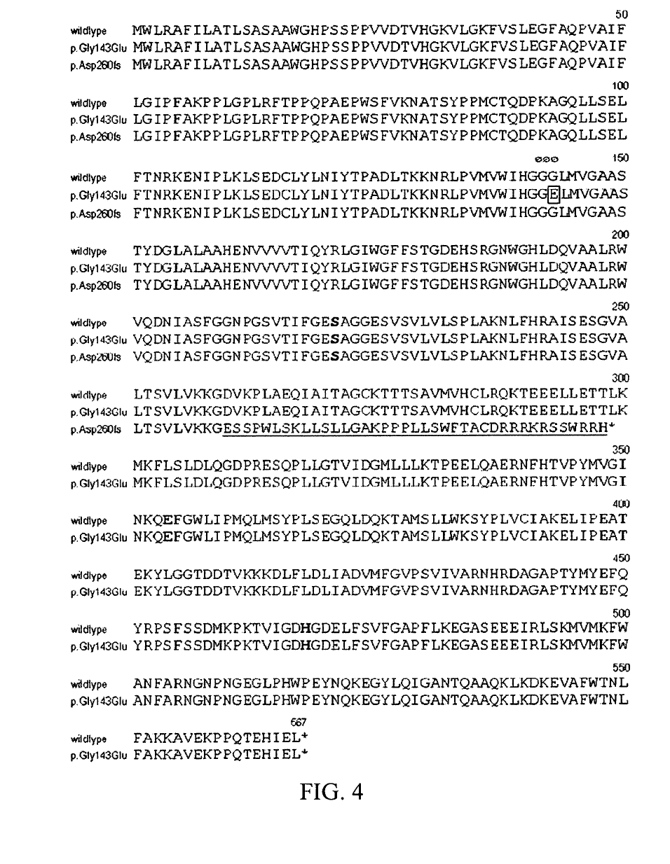

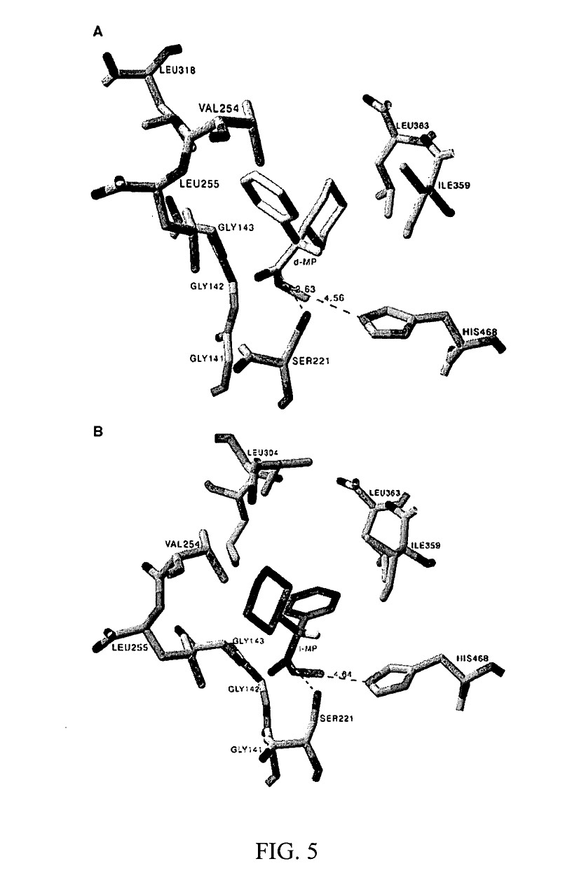

Glycine at position 143 of hCES1 is critical for hCES1 protein function. For hCES1, the corresponding active site triad residues are serine 221 (S), glutamic acid 354 (E), and histidine 468 (H; shown in bold in FIG. 4). A series of three consecutive glycine residues are also located in the active site of hCES1 (Gly141-143) and create what is referred to as an “oxyanion hole.” The oxyanion hole is thought to stabilize substrate-enzyme intermediates via hydrogen bonds formed with the oxyanion form of the carbonyl oxygen and, thus, would be fundamental to proper hCES1 function.¹⁹ The catalytic triad and oxyanion hole are evolutionarily conserved both across species (fish to humans) and within related serine hydrolases.¹⁹ FIG. 5 illustrates the location of Gly143 relative to the binding site of methylphenidate. When the glycine in hBuChE analogous to Gly143 in hCES1 was mutated both substrate affinity and catalysis were markedly reduced or abolished²⁰. This indicates that mutation of Gly143 to glutamic acid (p.Gly143Glu) may result in dysfunctional hCES1 due to disruption of the oxyanion hole.

Similarly, the 12785T>del mutation (p.Asp260fs) causes significant change in hCES1. For example, this frameshift mutation not only causes an early truncation and alteration of residues 260-299, but additionally eliminates two of three of the triad residues as well as other residues shown in FIG. 5 which may be critical for protein function. As shown in FIG. 4, in p.Asp260fs the subsequent frameshift and early truncation as a result of deletion of nucleotide 780 results in a protein missing two of the three conserved catalytic triad residues.

The data presented herein indicates that either or both of the polymorphisms presented herein are likely to cause significant loss of hCES1 activity. The slow metabolizer was heterozygous for both mutations and each mutation occurred on a different allele. Due to the frequencies of the each of the identified SNPs, having one of these SNPs on both alleles would be expected to be a rare occurrence. Nevertheless, the serendipitous identification of the slow metabolizer presented herein was critical for discovering two SNPs.

### III. INDIVIDUALIZATION OF THERAPIES

Carboxylesterase-1 is critical for the function and metabolism of many known compounds in humans and non-human animals. Thus, the presence or absence of one or more polymorphisms of the present invention may be used to “individualize” or modify a therapy for a subject or patient based on the sensitivity of the subject to a therapeutic due to the presence or absence of a polymorphism of the present invention.

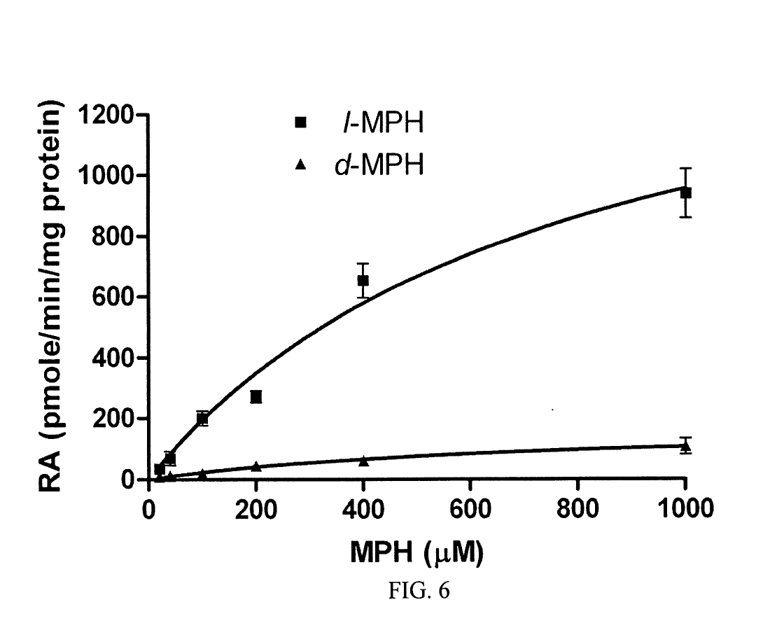

The hCES1 enzyme catalyzes the hydrolysis of drugs from numerous classes. The hydrolysis generally produces inactive metabolite(s) (e.g., MPH and cocaine).^{1,9} However, hCES1 is also known to be involved in the generation of active metabolites (e.g., conversion of heroin to monoacetylmorphine and morphine)^{1,2}, or the activation of prodrugs such as the angiotensin converting enzyme (ACE) inhibitors quinapril and benazepril.¹⁸ Although some overlap exists between CES1 and a related isoform CES2, CES1 is the isoform that mediates transesterification reactions.¹¹ The observation that the slow metabolizer was unable to form ethylphenidate when methylphenidate and ethanol were administered together further highlights the dysfunctional nature of the hCES1 variants. With regard to drugs of abuse, the existence of an unrecognized hCES1 deficiency could potentially lead to idiosyncratic toxicities and/or fatal exposures interpreted as accidental drug overdoses on the basis of antemortem or postmortem blood concentrations. Furthermore, these hydrolytic reactions can proceed on a stereoselective basis resulting in a distortion of the anticipated isomeric disposition of a racemic compound following the administration of a medication such as dl-MPH (FIG. 6).⁸

In certain embodiments, evaluating the presence or absence of a polymorphism of the present invention may be used to individualize a therapy and/or determine the sensitivity of a subject to a compound. The compound may be an illicit drug, heroin, cocaine, an Opioid, meperidine (also referred to as: isonipecaine; lidol; pethanol; piridosal; Algil®; Alodan®; Centralgin®; Demerol®; Dispadol®; Dolantin®; Dolargan® Dolestine®; Dolosal®; Dolsin®; Mefedina®), a dopaminergic or noradrenergic drug, methylphenidate (Ritalin®), an ACE Inhibitor, quinapril, enalapril, benzapril, imidapril, delapril, pemocapril, cilazapril, an anesthetic, lidocaine, a toxin, a chemical warfare agent, sarin nerve gas, soman, tabun, an insecticide, an organophosphate insecticide, lovastatin, an antiviral drug, oseltamivir, an anti-cancer drug, or ininotecan (CPT-11).

Methylphenidate is the most common pharmacologic agent used to treat attention-deficit hyperactivity disorder which afflicts school-age children with an estimated worldwide prevalence of 8-12%.^{3,4} Significant interindividual variability in MPH pharmacokinetics and pharmacodynamics is well recognized yet remains unexplained. The most common formulation of methylphenidate is the racemic mixture of d-threo-(R,R)- and l-threo-(S,S)-methylphenidate (MPH) enantiomer,⁵ with the d-isomer regarded as the active therapeutic isomer.⁶ The primary metabolic pathway governing the metabolism of MPH is rapid deesterification to the inactive metabolite, ritalinic acid (FIG. 1).⁷ This process is mediated by hCES1.⁸ Additionally, Sun and coworkers demonstrated in vitro that this hydrolytic process is highly enantioselective in that the catalytic efficiency of hCES1 is up to 6-fold higher for l-MPH than d-MPH.⁸ Furthermore, pharmacokinetic studies that have measured both isomers have consistently shown that the l-isomer is present at only 1-15% of the blood concentration of d-MPH.⁹ Additionally, the plasma half-life (t_{1/2}) of d-MPH is markedly longer than that of l-MPH.⁵

Based on these observations, the present invention permits one to establish a drug metabolism profiles for each drug and CES1 or variants thereof. By examining the CES1 gene or protein of the subject involved, one can then predict which drugs will be effective in the subject (if at all), and at which doses. Cells lines, described elsewhere in this application, have proven quite useful in conducting such analyses.

For example, in the course of conducting a randomized three-way crossover study of orally administered, racemic MPH at a single dose of 0.3 mg/kg aimed at investigating the interaction of MPH and ethanol in normal volunteers, a white male subject was identified with a highly unusual phenotype relative to MPH disposition suggestive of a metabolic deficiency in the ability to metabolize MPH.¹⁰ The design of the study is described elsewhere and included multiple blood sampling and repeated hemodynamic measurements throughout the study day.¹⁰ The Examples utilize data from only one of the 3 phases studied, when subjects received MPH only.

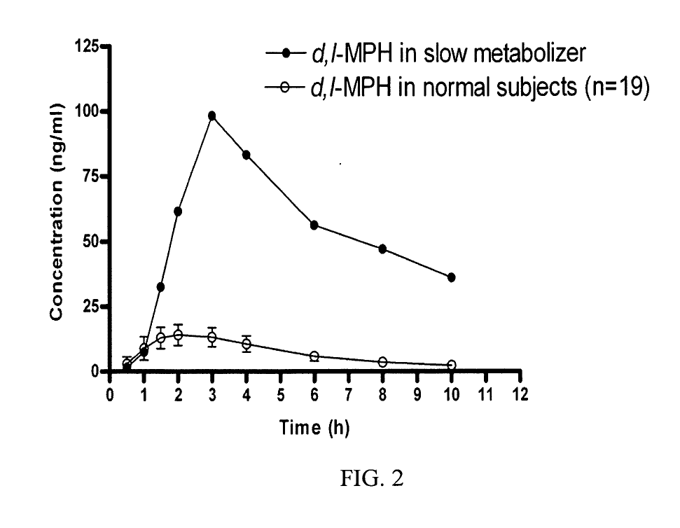

The subject appeared to be a slow metabolizer off MPH and displayed markedly elevated values for every measured pharmacokinetic parameter relative to the remaining 19 study subjects. When examining combined d- and l-MPH concentrations the magnitude of difference between the slow metabolizer and the remaining subjects is readily apparent (FIG. 2), wherein the observed maximum plasma concentration (Cₘₐₓ) is approximately 7-fold higher than the mean Cₘₐₓ determined for the other subjects. Differences in the concentration of the active d-MPH isomer in this subject versus study means (±SD) from all other subjects were as follows: the area under the plasma concentration-time curve extrapolated to infinity (AUC_{inf}) was 209 ng/ml·hr versus 83±22; the Cₘₐₓ was 37 ng/ml versus 15±3; the time to attain Cₘₐₓ (Tₘₐₓ) was 3.0 hr versus 2.3±0.8; and the plasma half life of elimination (t_{1/2}) was 5.4 hr versus 2.8±0.4 hr.⁹ An unprecedented observation was the extraordinarily high concentrations of the l-MPH species relative to d-MPH in the subject (Cₘₐₓ=62 ng/ml), a value approximately 100-fold higher than the mean concentrations for his study peers (FIG. 2) and literature values.^{8,10} Finally, the co-administration of methylphenidate with ethanol normally results in the hCES1 mediated formation of the transesterification metabolite ethylphenidate. Detectable concentrations of ethylphenidate were present in every subject except the slow metabolizer.

### IV. METHODS FOR DETECTION OF CARBOXYLESTERASE-1 POLYMORPHISMS

The presence or absence of one or both of the 12754T>del or Gly143Glu (9486G>A) polymorphisms in the CES1 gene may be evaluated using various techniques. For example, the carboxylesterase-1 gene may be cloned and sequenced to determine the presence or absence of a single nucleotide polymorphism. In certain embodiments, real-time PCR may be used to detect a single nucleotide polymorphism of the present invention. In other embodiments, techniques including PCR, multiplex PCR, gel electrophoresis, sequencing, hybridization with a probe specific for a single nucleotide polymorphism, restriction endonuclease digestion, primer extension, microarray or gene chip analysis, mass spectrometry, or a DNAse protection assay may be used for detecting a polymorphism of the present invention.

A. Real-Time PCR (rtPCR)

The presence of absence of polymorphisms of the present invention may be detected using real-time PCR. Real-time PCR typically utilizes fluorescent probes for the selective detection of the polymorphisms. Various real-time PCR testing platforms that may be used with the present invention include: 5′ nuclease (TaqMan® probes), molecular beacons, and FRET hybridization probes. These detection methods rely on the transfer of light energy between two adjacent dye molecules, a process referred to as fluorescence resonance energy transfer (see, e.g., Espy et al (2006) Clin Microbiol Rev. 2006 January; 19(1): 165-256 for a review of various rtPCR approaches that may be used with the present invention).

1. 5′ Nuclease Probes

In certain embodiments, a 5′ nuclease probe may be used to detect a polymorphism of the present invention. 5′ nuclease probes are often referred to by the proprietary name, TaqMan® probes. A TaqMan® probe is a short oligonucleotide (DNA) that contains a 5′ fluorescent dye and 3′ quenching dye. To generate a light signal (i.e., remove the effects of the quenching dye on the fluorescent dye), two events must occur. First, the probe must bind to a complementary strand of DNA, e.g., at about 60° C. Second, at this temperature, Taq polymerase, which is commonly used for PCR, must cleave the 5′ end of the TaqMan® probe (5′ nuclease activity), separating the fluorescent dye from the quenching dye.

In order to differentiate a single nucleotide polymorphism from a wild-type sequence in the DNA from a subject, a second probe with complementary nucleotide(s) to the polymorphism and a fluorescent dye with a different emission spectrum are typically utilized. Thus, these probes can be used to detect a specific, predefined polymorphism under the probe in the PCR amplification product. Two reaction vessels are typically used, one with a complementary probe to detect wild-type target DNA and another for detection of a specific nucleic acid sequence of a mutant strain. Because TaqMan® probes typically require temperatures of about 60° C. for efficient 5′ nuclease activity, the PCR may be cycled between about 90-95° C. and about 60° C. for amplification. In addition, the cleaved (free) fluorescent dye can accumulate after each PCR temperature cycle; thus, the dye can be measured at any time during the PCR cycling, including the hybridization step. In contrast, molecular beacons and FRET hybridization probes typically involve the measurement of fluorescence during the hybridization step.

Genotyping for the 12754T>del (“p.Asp260fs”) or Gly143Glu (9486G>A, “p.Gly143Glu”) in the carboxylesterase-1 gene may be evaluated using the following (5′ endonuclease probe) real-time PCR technique. Genotyping assays can be performed in duplicate and analyzed on a Bio-Rad iCycler Iq® Multicolor Real-time detection system (Bio-Rad Laboratories, Hercules, Calif.). Real-time polymerase chain reaction (PCR) allelic discrimination assays to detect the presence or absence of specific single nucleotide polymorphisms in the CES1 gene, p.Gly143Glu (genomic: nt 9486; Cdna: nt 428) and P.Asp260fs (genomic: nt 12754; Cdna: nt 780), may utilize fluorogenic TaqMan® Probes. The following primer sequences may be used:

| | |
|---|---|
| p.Gly143Glu forward: | |
| 5′-CCCAGGTGATGGTGTGGAT-3′ | (SEQ ID NO: 1) |
| p.Gly143Glu reverse: | |
| 5′-GCCAGCCCATCATAGGTTGA-3′ | (SEQ ID NO: 2) |
| p.Gly143Glu Probe, Vic labeled: | |
| 5′-CCATCAGCCCCCCTC-3′ | (SEQ ID NO: 3) |
| p.Gly143Glu Probe, Fam labeled: | |
| 5′-CCATCAGCTCCCCTC-3′ | (SEQ ID NO: 4) |
| p.Asp260fs forward: | |
| 5′-TGGCCCTCACTTCTGTTCTG-3′ | (SEQ ID NO: 5) |
| p.Asp260fs reverse: | |
| 5′-CCAGCCGGAGACCTACCT-3′ | (SEQ ID NO: 6) |
| p.Asp260fs Probe, Vic labeled: | |
| 5′-AAAGGTGATGTCAAGCC-3′ | (SEQ ID NO: 7) |
| p.Asp260fs Probe, Fam labeled: | |
| 5′-AAAGGTGAGTCAAGCC-3′ | (SEQ ID NO: 8) |

Real-time PCR amplifications may be carried out in a 10 μl reaction mix containing 5 ng genomic DNA, 900 Nm of each primer, 200 Nm of each probe and 5 μl of 2×TaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters may be: 95° C. for 10 min, followed by 50 cycles of 92° C. for 15 sec and 60 C.° for 1 min. Real-time fluorescence detection can be performed during the 60° C. annealing/extension step of each cycle. The IQ software may be used to plot and automatically call genotypes based on a two parameter plot using fluorescence intensities of FAM and VIC at 49 cycles.

2. Molecular Beacons

Molecular beacons are another real-time PCR approach which may be used to identify the presence or absence of a polymorphism of the present invention. Molecular beacons are oligonucleotide probes that are labeled with a fluorescent dye (typically on the 5′ end) and a quencher dye (typically on the 3′ end). A region at each end of the molecular beacon probe is designed to be complementary to itself, so at low temperatures the ends anneal, creating a hairpin structure. This hairpin structure positions the two dyes in close proximity, quenching the fluorescence from the reporter dye. The central region of the probe is designed to be complementary to a region of a PCR amplification product. At higher temperatures, both the PCR amplification product and probe are single stranded. As the temperature of the PCR is lowered, the central region of the molecular beacon probe may bind to the PCR product and force the separation of the fluorescent reporter dye from the quenching dye. Without the quencher dye in close proximity, a light signal from the reporter dye can be detected. If no PCR amplification product is available for binding, the probe can re-anneal to itself, bringing the reporter dye and quencher dye into close proximity, thus preventing fluorescent signal.

Two or more molecular beacon probes with different reporter dyes may be used for detecting single nucleotide polymorphisms. For example, a first molecular beacon designed with a first reporter dye may be used to indicate the presence of a SNP and a second molecular beacon designed with a second reporter dye may be used to indicate the presence of the corresponding wild-type sequence; in this way, different signals from the first and/or second reporter dyes may be used to determine if a subject is heterozygous for a SNP, homozygous for a SNP, or homozygous wild-type at the corresponding DNA region. By selection of appropriate PCR temperatures and/or extension of the probe length, a molecular beacons may bind to a target PCR product when a nucleotide polymorphism is present but at a slight cost of reduced specificity. Molecular beacons advantageously do not require thermocycling, so temperature optimization of the PCR is simplified.

3. FRET Hybridization Probes

FRET hybridization probes, also referred to as LightCycler® probes, may also be used to detect a polymorphism of the present invention. FRET hybridization probes typically comprise two DNA probes designed to anneal next to each other in a head-to-tail configuration on the PCR product. Typically, the upstream probe has a fluorescent dye on the 3′ end and the downstream probe has an acceptor dye on the 5′ end. If both probes anneal to the target PCR product, fluorescence from the 3′ dye can be absorbed by the adjacent acceptor dye on the 5′ end of the second probe. As a result, the second dye is excited and can emit light at a third wavelength, which may be detected. If the two dyes do not come into close proximity in the absence of sufficient complimentary DNA, then FRET does not occur between the two dyes. The 3′ end of the second (downstream) probe may be phosphorylated to prevent it from being used as a primer by Taq during PCR amplification. The two probes may encompass a region of 40 to 50 DNA base pairs.

FRET hybridization probe technology permits melting curve analysis of the amplification product. If the temperature is slowly raised, probes annealing to the target PCR product will be reduced and the FRET signal will be lost. The temperature at which half the FRET signal is lost is referred to as the melting temperature of the probe system. A single nucleotide polymorphism in the target DNA under a hybridization FRET probe will still generate a signal, but the melting curve will display a lower Tm. The lowered Tₘ can indicate the presence of a specific polymorphism. The target PCR product is detected and the altered Tm informs the user there is a difference in the sequence being detected. Like molecular beacons, there is not a specific thermocycling temperature requirement for FRET hybridization probes. Like molecular beacons, FRET hybridization probes have the advantage of being recycled or conserved during PCR temperature cycling, and a fluorescent signal does not accumulate as PCR product accumulates after each PCR cycle.

B. Primer Extension

Primer extension is another technique which may be used according to the present invention. A primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present. For example, U.S. Ser. No. 10/407,846, which is which is hereby incorporated by reference, describes a form of primer extension.

C. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

D. Sequencing

DNA sequencing may be used to evaluate a polymorphism of the present invention. For example, Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

E. Mass Spectrometry

Mass spectrometry may also be used to detect a polymorphism of the present invention. By exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). Methods of mass spectroscopy that may be used with the present invention include: ESI, ESI tandem mass spectroscopy (ESI/MS/MS), Secondary ion mass spectroscopy (SIMS), Laser desorption mass spectroscopy (LD-MS), Laser Desorption Laser Photoionization Mass Spectroscopy (LDLPMS), and MALDI-TOF-MS.

F. Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

G. Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments, as described above, also contemplate the use of Taqman® and Molecular Beacon® probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples may be detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P may be used. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

H. Amplifying a Target Sequence

In a particular embodiment, it may be desirable to amplify the target sequence before evaluating the SNP. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with XbaI, and ligate on M13-universal primers with an XbaI over hang, followed by amplification of the genomic DNA with an M13 universal primer. Only a small percentage of the total DNA would be amplified (the restriction fragments that were less than 1000 bases). One would then use labeled primers that correspond to a SNP are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

### V. KITS

All the essential materials and reagents required for detecting nucleic acid mutations in a sample may be assembled together in a kit. This generally will comprise a primer or probe designed to hybridize specifically to, upstream and/or downstream of target nucleotides of the polymorphism of interest. The primer or probe may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10× buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products. In various embodiments, the kit may further comprise one or more reagents to test for a CYP2D6 polymorphism and/or CYP2D6 function; in these embodiments, one may more effectively individualize a therapy for ADHD.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### VI. EXPRESSION CONSTRUCTS AND PRODUCTION OF CELL LINES

In one aspect, the present invention provides for the production of cells and cell lines that express wild-type CES1 in normal, reduced, or increased levels as compared to wild-type/non-pathologic cells, as well as for the expression of mutant CES1 molecules, such as those described herein. The techniques for producing such cells are well known in the art and generally follow standard methods of recombinant cell production, such as those described Sambrook et al. Some of the aspects of recombinant host cell production are described below in greater detail.

A. Expression Constructs

Expression constructs are nucleic acids that contain regulatory elements facilitating the expression of a given gene product, and a nucleic acid segment encoding that gene product. Often, such constructs are included in “vectors,” carrier nucleic acid molecules into which an expression cassette can be inserted for introduction into a cell where it can be replicated. A nucleic acid sequence can be “exogenous,” which means that it is foreign to the cell or vector into which it is being introduced, or the sequence is homologous to a sequence in the cell or vector, but in a position within the host cell genome or vector backbone in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

1. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 by upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cells, promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference).

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous to the vector or host cell.

The promoter may be a strong, constitutive promoter. Such promoters are useful in the context of high level or “overexpression” of a target protein. The cytomegalovirus immediate early (CMV IE) promoter is one such example. Other constitutive promoters that are useful in the present invention include SV40. Another class of promoters are the tissue-specific promoters or elements. These promoters show preferential or selective expression in certain cell types. Other promoters suitable for use with the present invention include α-myosin heavy chain (heart), α-fetoprotein (liver), albumin (liver), thyroglobulin (thyroid), enolase (brain), CC10 (lung), keratin (epidermis) and (β-lactoglobulin (mammary gland).

Yet another type of promoter that may be used is an inducible promoter. Inducible promoters are activated by an exogenous signal that can be provided at the discretion of the user. An example of an inducible promoter is the Tet-On®/Tet-Off® Systems (Clontech). Both Tet-On® and Tet-Off® use a chimeric transactivator to activate transcription of the gene of interest from a silent promoter. The transactivator, either tTA or rtTA, is expressed in a host cell from a constitutive or tissue specific promoter. In the Tet-Off® system, tTA binds to the Tet Response Element (TRE) in the silent promoter and activates transcription in the absence of the inducer, doxycycline. In the Tet-On® System, rtTA binds to the TRE and activates transcription in the presence of doxycycline. Again, such promoter can achieve “high level” expression that exceeds, sometimes by many-fold, that seen in “normal” or wild-type cells.

In the present invention, hypertrophic promoters are those promoters believed to be particularly active cells exhibiting hypertrophic gene expression patterns, and thus useful in the screening assays described herein. These promoters include ANF, α-skeletal actin, myoglobin, α-myosin heavy chain, β-myosin heavy chain, NFAT, MEF-2, SERCA and any other gene known to be up- or down-regulated in the hypertrophic heart.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

7. Origins of Replication

In order to propagate a vector in a host cell, the vector may contain one or more origins of replication sites (often termed “ori”), which is a specific nucleic acid sequence at which replication is initiated. Alternatively, an autonomously replicating sequence (ARS) can be employed if the host cell is yeast.

B. Methods of Gene Transfer

1. Non-Viral Delivery

As part of the present invention, it will be necessary to transfer various genetic constructs into cells, both for transient expression and stable transformation. Suitable methods for nucleic acid delivery for use with the current invention are believed to include virtually any method by which a nucleic acid (e.g., DNA) can be introduced into a cell, as described herein, or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome-mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods.

2. Viral Methods

The ability of certain viruses to infect cells and express transgenes transiently or stably have made them attractive candidates for the transfer of foreign nucleic acids into cells. Thus, the present invention may take advantage of viral vectors to deliver reporter gene constructs or other genes relevant to cardiac drug screening methods. Non-limiting examples of virus vectors that may be used to deliver a nucleic acid of the present invention are adenovirus, AAV, retrovirus, vaccinia virus, sindbis virus, cytomegalovirus and herpes simplex virus.

In addition, a nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### VII. SCREENING METHODS

The present invention also provides for cell-based screening methods to identify compounds that are beneficial substrates for, or modulators of, CES1. These assays may comprise random screening of large libraries of candidate substances; alternatively, the assays may be used to focus on particular classes of compounds selected with an eye towards structural attributes that are believed to make them more likely to modulate CES1 function or to be a substrate therefor.

It will, of course, be understood that all the screening methods of the present invention are useful in themselves notwithstanding the fact that effective candidates may not be found. The invention provides methods for screening for such candidates, not solely methods of finding them.

A. Candidate Substances

As used herein the term “candidate substance” refers to any molecule that may potentially inhibit cardiac hypertrophy. The candidate substance may be a protein or fragment thereof, a small molecule, or even a nucleic acid molecule. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

B. Assay Formats

In a first assay format, one will examine the effects of a candidate substance on promoter expression. Typically, this will involve the use of a transgene comprising a coding region for a reporter linked to the promoter of interest. The reporters may provide visual readouts (colored enzyme products, fluorescent signals) that can be monitored (optimally in a high-throughput fashion) by automated optical systems. Typical assays involve multi-well microtitres plates.

A second assay format looks at rate or extent of substrate turnover, where the substrate is the candidate substance, or where the candidate substance is used to modulate the rate or extent of turnover of a known substrate. This may also involve examining the localization of a substrate or product, such as areas within a cell or external thereto.

In a third assay format, one will examine the isomerization or other structural modification of a substrate or candidate substance, the former being assessed in the present of a candidate substance. Formats looking at CES1 activity may involve the use of normal, wild-type overexpressed, or mutant CES1 enzymes.

C. Reporter Genes

As discussed above, some assays will utilize various screenable markers genes. Such marker or “reporter” genes permit assaying of gene expression levels by looking for a signal. Suitable reporter genes include enzymes that produce a screenable product. One such enzyme is β-galactosidase, when used in the presence of specific substrates. This enzyme produces a blue product that is directly proportional to the amount of enzyme present in the sample. The product can be measured optically. Another enzyme useful in screening activity is chloramphenicol acetyl transferase, or CAT.

In another embodiment, the screenable marker may be a fluorescent chemilluminescent molecule, such as firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized as fluorescence following illumination by particular wavelengths of light.

Other screenable molecules include β-glucuronidase, enhanced GFP, blue fluorescent protein, secreted alkaline phosphatase, Renilla luciferase and xylene oxidase.

D. Product Separation Technologies

1. Chromatography

Any of a wide variety of chromatographic procedures may be employed according to the present invention. For example, thin layer chromatography, gas chromatography, high performance liquid chromatography, paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

2. Mass Spectroscopy

As discussed above, by exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while quantitative methods are being developed using matrix assisted laser desorption/ionization (MALDI) followed by time of flight (TOF) MS (Bucknall et al., 2002; Mirgorodskaya et al., 2000; Gobom et al., 2000). Methods of mass spectroscopy that may be used with the present invention include ESI, ESI tandem mass spectroscopy (ESI/MS/MS), secondary ion mass spectroscopy (SIMS), laser desorption mass spectroscopy (LD-MS), laser desorption laser photoionization mass spectroscopy (LDLPMS), and MALDI-TOF-MS.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Identification of CES1 genetic variants. Total genomic DNA was extracted from whole blood for CES1 DNA sequence analysis. DNA sequencing, initial SNP identification, and mutant sequence verification was performed by SeqWright, Inc. Laboratories (Houston, Tex.). Fifty-two custom primers were used in the bi-directional sequencing of all 14 CES1 exons, including 50-200 bp of flanking intronic region at each exon (GenBank accession numbers: genomic reference, AB119997; cDNA, AB119995). Introns were not investigated further. Sequence at the 5′ end extended ˜12 bp upstream of exon 1 and at the 3′ end ˜13 bp downstream of exon 14. Additional primer sets used to verify the two described mutations:

| | |
|---|---|
| Exon4forward: | |
| 5′-TGATGGGAGTGTCCTCCCGAAG-3′ | (SEQ ID NO: 9) |
| Exon4reverse: | |
| 5′-GGGTAGGTAGTGTGTCCAATTAC-3′ | (SEQ ID NO: 10) |
| Exon6forward: | |
| 5′-AGGAAGACTTCCACCTCCTTG-3′ | (SEQ ID NO: 11) |
| Exon6reverse: | |
| 5′-AGGAGTGTGGTCACACAGATAG-3′ | (SEQ ID NO: 12) |

Sequence delineation and basecalling was performed using automated fluorescent DNA sequencers (ABI model 3730x1; Applied Biosystems, Foster City, Calif.). Cycling conditions were as follows: 30 cycles of 95° C. for 30 sec, 50-60° C. (depending on Tm) for 30 sec, and 70° C. for 1 min.

All procedures were approved by the Medical University of South Carolina Institutional Review Board and were performed only after obtaining written Informed Consent. In the case of additional DNA samples obtained from additional subjects for purposes of determining SNP frequency in the general population, all procedures were approved by the University of Florida Institutional Review Board and written informed consent was obtained.

Genotyping for CES1 Variants and Determination of SNP Frequency. Genotyping assays were performed in duplicate and analyzed on a Bio-Rad iCycler iQ® Multicolor Real-time detection system. Real-time polymerase chain reaction (PCR) allelic discrimination assays were designed using Assay-by-Design service and the specific variants in the CES1 gene, p.Gly143Glu (genomic: nt 9486; cDNA: nt 428, dbSNP ss number: 99307125) and p.Asp260fs (genomic: nt 12754; cDNA: nt 780, dbSNP ss number: 99307126), were identified using fluorogenic TaqMan® Probes. The sequences of primers and probes were as follows:

| | |
|---|---|
| p.Gly143Glu forward: | |
| 5′-CCCAGGTGATGGTGTGGAT-3′ | (SEQ ID NO: 1) |
| p.Gly143Glu reverse: | |
| 5′-GCCAGCCCATCATAGGTTGA-3′ | (SEQ ID NO: 2) |
| p.Gly143Glu Probe, Vic labeled: | |
| 5′-CCATCAGCCCCCCTC-3′ | (SEQ ID NO: 3) |
| p.Gly143Glu Probe, Fam labeled: | |
| 5′-CCATCAGCTCCCCTC-3′ | (SEQ ID NO: 4) |
| p.Asp260fs forward: | |
| 5′-TGGCCCTCACTTCTGTTCTG-3′ | (SEQ ID NO: 5) |
| p.Asp260fs reverse: | |
| 5′-CCAGCCGGAGACCTACCT-3′ | (SEQ ID NO: 6) |
| p.Asp260fs Probe, Vic labeled: | |
| 5′-AAAGGTGATGTCAAGCC-3′ | (SEQ ID NO: 7) |
| p.Asp260fs Probe, Fam labeled: | |
| 5′-AAAGGTGAGTCAAGCC-3′ | (SEQ ID NO: 8) |

Real-time PCR amplifications were carried out in a 10 μl reaction mix containing 5 ng genomic DNA, 900 nM of each primer, 200 nM of each probe and 5 μl of 2×TaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters were: 95° C. for 10 min, followed by 50 cycles of 92° C. for 15 sec and 60 C.° for 1 min. Real-time fluorescence detection was performed during the 60° C. annealing/extension step of each cycle. The IQ software was used to plot and automatically call genotypes based on a two parameter plot using fluorescence intensities of FAM and VIC at 49 cycles.

Genomic DNA from 13 additional participants (10 male and 3 female Caucasians) from the same pharmacokinetic study as the aberrant metabolizer was also screened for CES1 variants. These participants exhibited “normal” MPH concentrations consistent with those typically observed in research subjects and available patient studies. The aberrant metabolizer's biological parents were also screened for the CES1 variants. In order to estimate the allelic frequency of these CES1 variants in the general Caucasian population, a genomic DNA panel (HD100CAU) representing a cohort of 100 self-declared Caucasian individuals (51 males, 49 females) obtained through Coriell Cell Repositories (Coriell Institute, Camden, N.J.) was screened. Additionally, genomic DNA samples collected from various self-identified racial and ethnic groups during the course of participation in a large multi-center clinical study of combination antihypertensive treatment (INternational VErapamil SR/Trandolapril Study [INVEST]) were also available for analysis. This group consisted of 355 additional Caucasians subjects, 117 Blacks subjects, 299 subjects of Hispanic ethnicity, and 54 Asian individuals. The aberrant metabolizer, his parents, and study peers were not included in the calculation of allelic frequency since a priori information regarding their CES1 activity was known to investigators.

Analysis of Pharmacokinetic, Hemodynamic, and Statistical Data. Standard pharmacokinetic analyses were applied to the MPH data using WinNonLin software (Pharsight, Mountainview, Calif.) and are described in a previous publication.¹⁰ The AUC, Cₘₐₓ, and t_{1/2} were analyzed using the Extreme Studentized Deviate (ESD) single-outlier procedure¹² to determine whether the parameter measurements for the slow metabolizer constituted true statistical outliers. For each parameter, the ESD procedure confirmed the measurement as an outlier at the p<0.01 level. Because this procedure is known to be conservative, the data from the aberrant metabolizer were reassessed using a similar procedure with respect to the normal participants only.

To determine whether the slow metabolizer of MPH experienced significantly different pharmacodynamic effects as a result of elevated MPH blood concentrations, the inventor examined hemodynamic data that had been collected at 8 time points after MPH administration during the study.¹⁰ Beyond each subject's measurements of systolic blood pressure (SBP), diastolic blood pressure (DBP), and heart rate (HR), the mean arterial pressure (MAP) was also calculated according to the following formula: MAP=DBP+⅓(SBP−DBP).

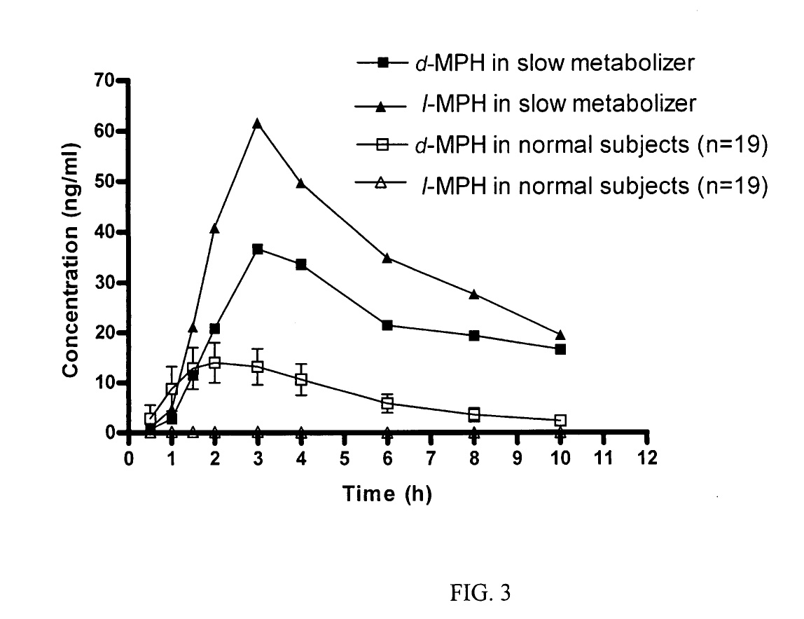

The inventor considered the set of measures for each endpoint to be a multivariate vector, and assessed the Mahalanobis distance for subject's measurements. The Mahalanobis distance, a measure of distance from a central multivariate mean relative to the variance of the endpoints, is useful for identifying outliers via referral to the F distribution. This analysis assumes no explicit model of time effects on the vital signs, but recognizes the potential correlation between the repeated measures over time on each participant, and tests for simultaneous differences from the mean over the set of time points while controlling the Type I error rate. For each of the four endpoint vectors, the inventor tested the a priori hypothesis that the slow metabolizer's measurements were outliers by comparison to Wilks's critical values (α=0.0125, critical value: 13.8).¹³ Because only testing outlier status for this single participant was tested, no Bonferroni corrections were made within participants; however, each test was performed at the Bonferroni-corrected α=0.0125 level to adjust for the four endpoints being tested. There were little differences in plasma MPH concentrations between the slow metabolizer and his study peers during the 0-1.5 h time frame (FIG. 6), which is likely due to a delay in tablet disintegration, dissolution and absorption. After this initial period MPH is subject to first-pass metabolism and hCES1 mediated stereoselective hydrolysis and the two concentration vs. time curves rapidly diverge from one another (FIGS. 2-3). Therefore, based on this finding and a visual examination of the data it was decided that a second analysis which excluded measurements taken during the 0-1.5 hr time points would be more appropriate.

The allele frequency was determined by gene counting. Additionally, genotype frequencies were tested within individual sexes, and ethnicity group for departure from Hardy-Weinberg Equilibrium by using Chi square test with one degree of freedom.

In vitro functional studies. There are two major advantages of utilizing Flp-In-293 cells as the parent cells to establish the cell line stably expressing hCES1.
1. Flp-In-293 cells exhibit extremely low background hCES1 expression and catalytic activity.

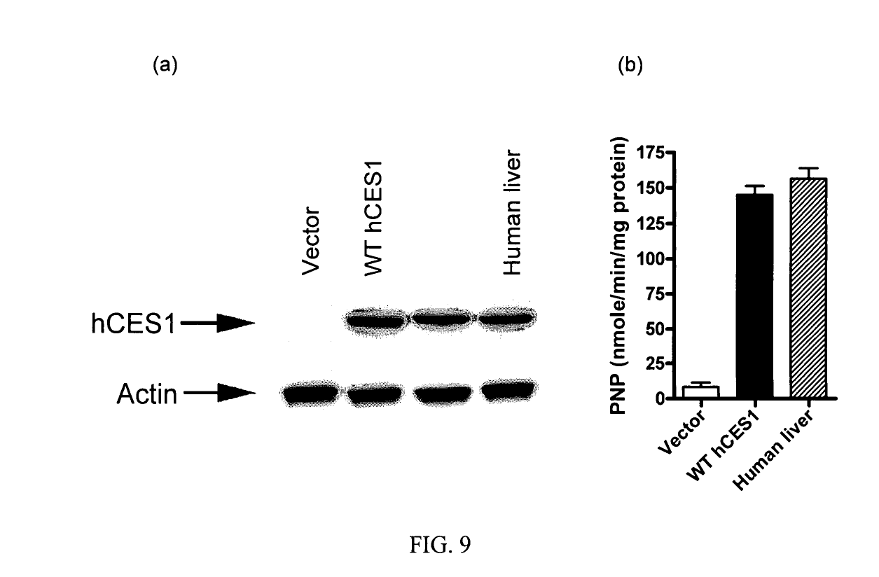

First, Flp-In-293 cells are human in origin (kidney) unlike most all other Flp-In cells available which have been developed from animal cell lines. Western blot studies performed in the inventor's laboratory demonstrated the intrinsic expression of hCES1 in the parent Flp-In-293 cells is essentially undetectable while the transfected cells display strong hCES1 expression to a degree that is similar to normal human liver tissues (FIG. 9A). Furthermore, the hydrolytic activity of non-transfected Flp-In-293 cells on a standard hydrolase substrate, PNPA, was determined to be extremely low. By comparison, the cells transfected with the CES1 gene encoding for the hCES1 enzyme exhibited remarkable hydrolytic activity comparable to normal human liver tissues (FIG. 9B).
2. The Flp-In System involves introduction of a Flp Recombination Target (FRT) site into the genome of the Flp-In cell lines including Flp-In-293 cells.

An expression vector containing CES1 is then integrated into the genome via Flp recombinase-mediated DNA recombination at the FRT site. The CES1 gene is inserted in exactly the same genomic site in each transfected cell and only a single copy of each gene is integrated in each cell. Thus, this model is composed of highly uniform cells that stably express hCES1.

Human CES1A1 cDNA cloned into a pCMV-SPORT6 vector was purchased from ATCC. A construct capable of expressing human CES1 in Flp-In™-293 cells (Invitrogen, Carlsbad, Calif.) was generated using a TA cloning strategy. Briefly, the human CES1A1 gene was amplified via PCR, inserted into pcDNA5/FRT/V5-His-TOPO® vector, and transformed into One Shot® TOP10 Chemically Competent E. coli cells (Invitrogen, Carlsbad, Calif.). The human CES1A1 gene was amplified via Taq polymerase (Takara EX Taq™ HS, Shiga, Japan) based PCR using the following primers:

| | |
|---|---|
| Forward: | 5′-GAACTGTCGCCCTTCCACGATG-3′ |
| Reverse: | 5′ CAGCTCTATGTGTTCTGTCTGGGG-3′ |

The PCR cycling parameters are as follows:

| Stage | Cycles | Temperature | Time | |
|---|---|---|---|---|
| 1 | 1 | 95° C. | 1 | minute |
| 2 | 30 | 95° C. | 30 | seconds |
| | | 55° C. | 30 | seconds |
| | | 72° C. | 2 | minutes |

| | | | |
|---|---|---|---|
| 3 | 1 | 72° C. | 10 minutes, hold at |
| | | | 4° C. |

The PCR product was visualized following 1% agarose gel electrophoresis. A single DNA band with the expected molecular weight of 1.72 kb was obtained. The DNA was gel-purified, and the desired sequence was confirmed by DNA sequencing using the primers described above and two additional customized primers (Forward: 5′-GTCCAGGACAACATTGCCAG-3′; Reverse: 5′-TGCTATCAAGTCCAGGAACAGG-3′). The PCR product was inserted into pcDNA5/FRT/V5-His-TOPO® vector, and transformed into One Shot® TOP10 Chemically Competent E. coli cells (Invitrogen®). After culture on LB plates containing 100 μg/ml ampicillin, ten transformants were chosen for an additional overnight culture in 5 ml of LB medium containing 100 μg/ml ampicillin. The plasmids were extracted using an Eppendorf® FastPlasmid™ Mini Kit (Fisher Scientific), and analyzed by use of restriction enzyme and DNA sequencing. The desired plasmid was designed such that the recombinant hCES1 would be expressed in mammalian cells with a V5-His tag to facilitate detection and purification. Additionally, another plasmid was generated using a site-directed mutagenesis assay to insert a stop codon after CES1A1 gene to yield the construct expressing hCES1 without V5-His tag. The sense mutagenic primer is: 5′-CAGAACACATAGAGCTGTAAGGGCGAGCTTGGTAC-3′ whereas the antisense mutagenic primer is: 5′-GTACCAAGCTCGCCCTTACAGCTCTATGTGTTCTG-3′. A construct of the CES1 mutations, p.Gly143Glu and was generated also using the site-directed mutagenesis assay. The mutagenic primers for p.Gly143Glu are listed below:

p.Gly143Glu Sense:
5′-TCCACGGAGGGGAGCTGATGGTGGG-3′
p.Gly143Glu Antisense:
5′-CCCACCATCAGCTCCCCTCCGTGGA-3′
p.Asp260fs Sense:
5′-TGGTGAAGAAAGGTGAGTCAAGCCCTTGGCTG-3′
p.Asp260fs Antisense:
5′-CAGCCAAGGGCTTGACTCACCTTTCTTCACCA-3′

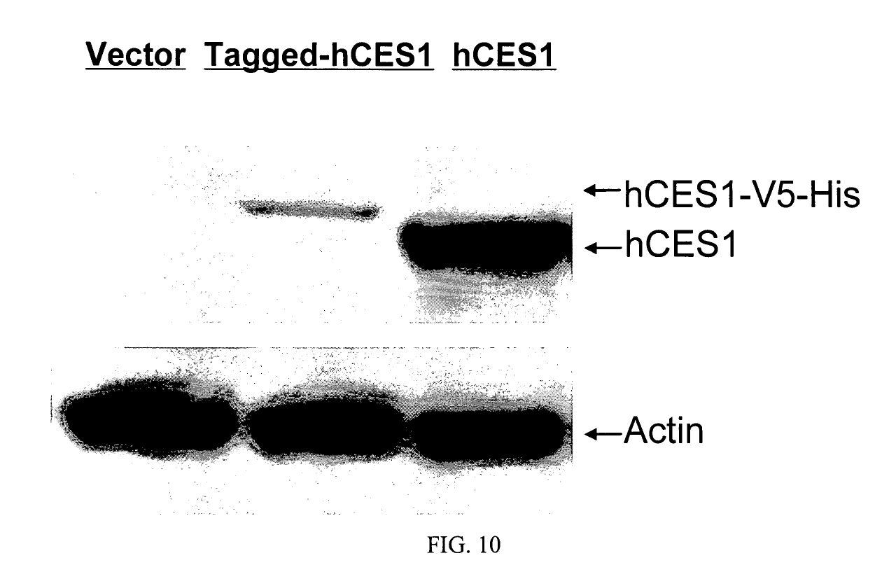

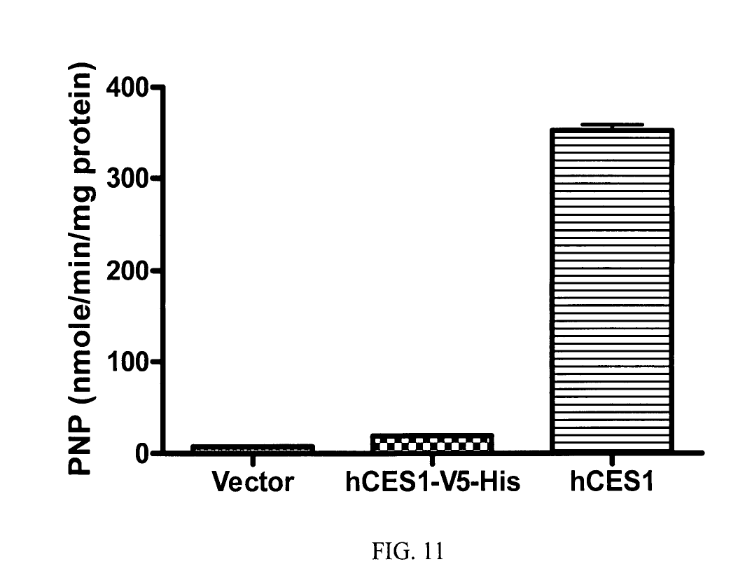

The desired plasmid was designed such that the recombinant CES1 would be expressed in mammalian cells with a V5-His tag to facilitate detection and purification. Additionally, another plasmid was generated by inserting a stop codon after CES1A1 gene to yield the constructs expressing CES1 without V5-His tag. Western blotting revealed that the detectable expression levels of V5-His tagged CES1 were significantly lower than that of untagged CES1, which may be caused by incorrect protein folding of tagged CES1 (FIG. 10). Furthermore, the enzymatic activity of the cells transfected with tagged hCES1 toward a standard esterase substrate p-nitrophenyl acet (PNPA) is significantly lower than that of untagged hCES1 (FIG. 11). Thus, only untagged CES1 and its associated mutants were used in the enzymatic hydrolysis study. Two constructs of CES1 mutations, p.Gly143Glu and p.Asp260fs, were generated using a site-directed mutagenesis assay. All constructs were subjected to DNA sequencing analysis to confirm that the desired plasmids were obtained. The identified CES1A1 plasmids (WT, p.Gly143Glu, and p.Asp260fs) were co-transfected with pOG44 plasmid at a ratio of 1:10 into Flp-In™-293 cells using Lipofectamine 200™ (Invitrogen, Carlsbad, Calif.). Additionally, the self-ligated pcDNA5/FRT/V5-His-TOPO® vector was included as a vector control. Twenty-four hours after transfection, cells were washed with PBS and fresh complete medium (Dulbecco's modified Eagle's medium containing 10% fetal bovine serum) was added. Forty-eight hours after transfection, cells were split at a 1:10 ratio and cultured at 37° C. until cell attachment was observed. Culture medium was then removed and replenished with complete medium supplemented with the selecting antibiotic hygromycin B (100 μg/ml). The cell lines stably expressing WT, p.Gly143Glu, and p.Asp260fs CES1 were obtained only after a minimum 3-week selection process employing hygromycin B. Each generated cell line was identified by both expression detection and enzymatic function assays.

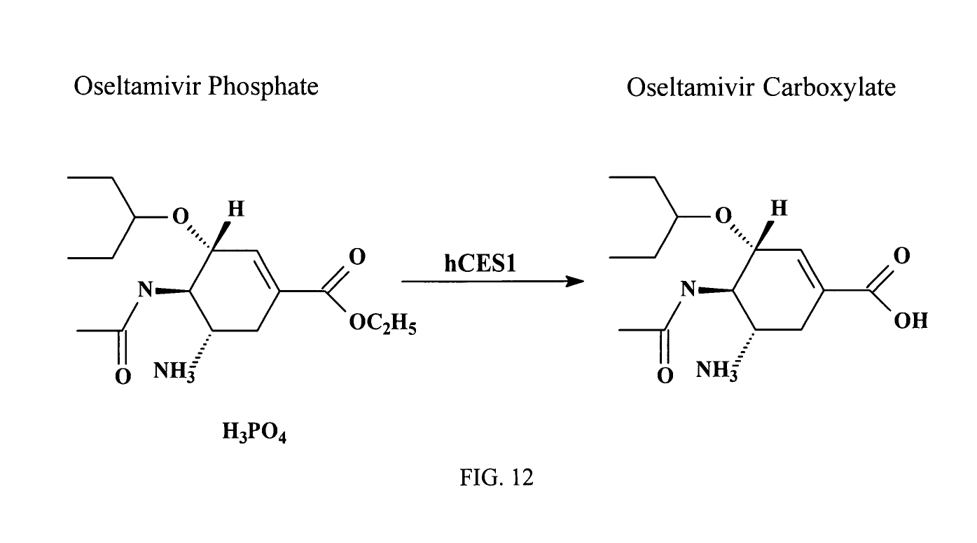

Enzymatic function study. Chosen substrates for the site-directed mutagenesis enzymatic studies were d- and l-MPH due to their role in the inventor's initial discovery of the SNPs described as well as almost exclusive dependence on hCES1 for deactivation (i.e., metabolism). Since hCES1 is also of critical importance in the activation of many prodrugs a representative substrate, oseltamivir phosphate ([OP] Tamiflu®) was chosen as an attractive substrate for investigation of the influence of mutated CES1 on it's metabolic activation via hCES1.²² In this instance, hCES1 is required to convert OP into oseltamivir carboxylate (OC) (FIG. 12). It is the liberated OC which exerts antiviral activity by inhibiting influenza virus neuraminidase. Furthermore, the active OC metabolite undergoes essentially no additional metabolism. Thus, as with MPH, biotransformation is almost entirely dependent on metabolic deesterification. Finally, OP was of additional interest due to its frequent use in the pediatric population and ongoing concerns over a substantial number of serious neuropsychiatric events associated with the drug. Of note, within the pharmacology review in the original New Drug Application (NDA) of OP²³ it was noted that juvenile rats had much difficulty hydrolyzing OP to form OC. Additionally, the report goes on to suggest most toxicities may be associated with higher prodrug exposure. These adverse effects were sufficient to prompt a “Dear Healthcare Provider” letter in November of 2006 that drew attention to accumulating reports of self-injury, delirium and other events in pediatric patients and accompanied a revised package insert. However, continued concerns over approximately 600 reports of adverse psychiatric reactions of this nature in pediatric patients since 1999 (75% from Japan) have resulted in a further review by an FDA panel and recommendations in late 2007 to Roche (which were accepted) to strengthen the cautionary notes in the package insert.

After attaining approximately 95% confluence, cells were rinsed and harvested in PBS containing 10 mM HEPES (pH 7.4). The cell suspension was then sonicated and the supernatant 9000 (S9) fraction was collected by centrifugation at 9000 g for 30 min at 4° C. The protein concentration was determined using a Pierce BCA assay kit (Rockford, Ill.).

The hydrolysis of PNPA was carried out in 96-well culture plates at 37° C. in a final volume of 200 μl. The S9 of cells expressing WT and mutant CES1 were diluted in reaction buffer (PBS containing 10 mM HEPES, pH 7.4) with a final S9 concentration of 20 μg/ml and preincubated at 37° C. for 10 min. The reaction was initiated by adding a range of concentrations of PNPA (20 μM-1000 μM). The formation of p-nitrophenol (PNP) from PNPA was determined by the absorbance at 405 nm following incubation at 37° C. for 10 min.

For the study of MPH hydrolysis, the reaction was conducted in 1.5 mL eppendorf tubes with a final S9 concentration of 0.5 mg/mL at a total volume of 100 μL. Prior to incubations, d- and l-MPH solutions were prepared freshly in 50 μL reaction buffer and then mixed with 50 μL of S9 with final substrate concentrations ranging from 20 μM to 1000 μM. After incubation at 37° C. for 2 hours, the reaction was terminated by adding 500 μl of methanol. The precipitated protein was then removed by centrifugation (20,000 g for 5 min at 4° C.). Concentrations of the primary phase I MPH metabolite produced via hydrolysis, ritalinic acid (RA), was determined utilizing an established HPLC method described below.

Both PNPA and MPH were spontaneously hydrolyzed in the reaction buffer. Thus, following the enzymatic assay, the values determined for spontaneous PNP and RA hydrolysis were subtracted from the total PNP and RA production. Notably, the empty vector transfected cells did not display any measurable catalytic activity or expression of CES1. All collected data were fit to the Michaelis-Menten equation, and kinetic parameters were calculated using nonlinear regression analysis with Graphpad Prism software (Graphpad Software Inc., San Diego, Calif.)

HPLC analysis. A high-performance liquid chromatography (HPLC) method was employed to determine RA concentrations based on the method described by Soldin and associates. The HPLC system consisted of an Agilent 1100 module, a C18 reversed-phase column (250×4.6 mm, 5 μm) preceded by a 4 mm×3 mm C18 guard column (Phenomenex, Torrance, Calif.), and a diode-array detector. The separation was performed using acetonitrile/20 mM KH₂PO₄, pH3.8 (16/84, v/v) with a flow rate of 1.0 mL/min and column temperature set at 40° C. The detection wavelength was set at 192 nm. RA was eluted at 7.6 min. The lower limit of quantification of RA was 0.2 μm.

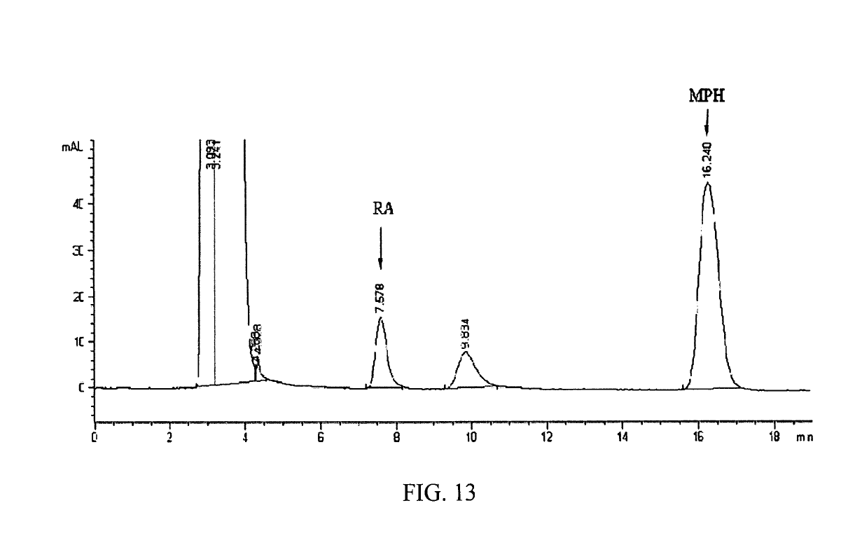

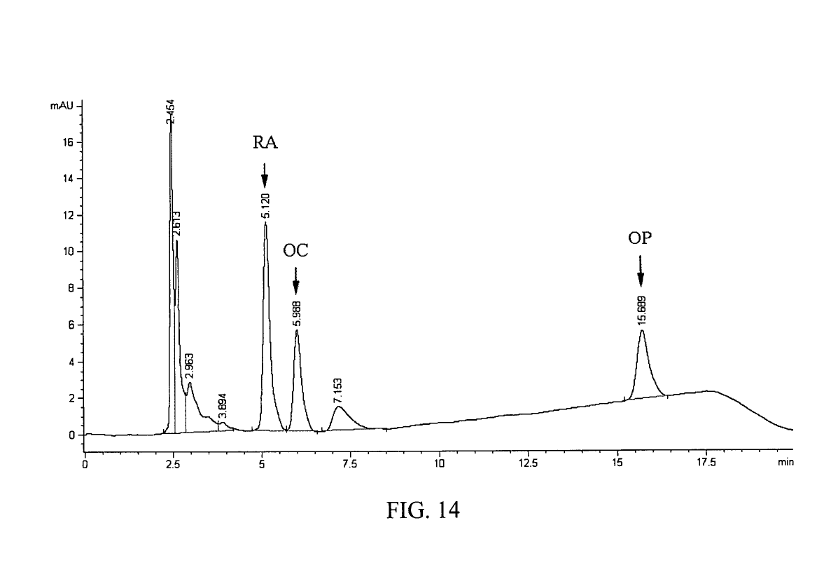

A typical chromatogram is shown in FIG. 13, where 100 μM of l-MPH were hydrolyzed by WT hCES1 s9 at 37° C. for 2 hours. To quantify the hydrolysis product of OP, OC, a HPLC method was established and validated. After incubation at 37° C. for 10 min, the OP hydrolysis reaction was terminated by adding 500 μA of methanol containing 40 μM RA as the internal standard. It was noted that RA, the primary hydrolytic product measured in the MPH assays was also found to serve as an excellent internal standard in this separately conducted OP/OC assay. The mixture was centrifuged to precipitate protein, and the supernatants were then analyzed utilizing an Agilent 1100 HPLC system equipped with a diode-array detector with the wavelength set at 220 nm. The mobile phase was a mixture of methanol and 20 mM KH₂PO₄ (pH2.5). A gradient elution was applied for the separation with the time program set as follows: from 0 to 4, methanol was 44% and increased to 50% from 4 to 14 min, then maintained at 50% until 16 min, where methanol was returned to the initial condition (44%). Ritalinic acid, OC, and OP were eluted at 5.1, 6.0, and 15.7 min, respectively, when the flow rate was set at 1 ml/min. The lower limit of quantification of OC was 0.25 μM. FIG. 14 is a typical chromatogram of 100 μM of OP hydrolyzed by WT hCES1 s9 at 37° C. for 10 min. The intra-day and inter-day relative standard deviations were determined to be less than 10% for both RA and OC assays.

### Example 2

### Two CES1 Gene Mutations Lead to Dysfunctional Carboxylesterase 1 Activity in Man: Clinical Significance and Molecular Basis

Pharmacokinetic Statistical Analysis. Based on the ESD analyses applied to the data, the inventor estimated the subject's AUC, Cₘₐₓ, and t_{1/2} values of d-MPH were 7.3, 4.9, and 5.2 standard deviations from the mean of the other 19 normal volunteers, respectively (Table 1). These results demonstrate that this aberrant metabolizer's individual key pharmacokinetic parameters (i.e., AUC_{inf}, Cₘₐₓ, t_{1/2}) were statistical outliers suggestive of a metabolic abnormality.

TABLE 1
Pharmacokinetic parameters in the aberrant
metabolizer versus the 19 study peers

| Pharmacokinetic | | | | |
|---|---|---|---|---|
| Parameter | Mean (±SD) | Outlier | ESD Statistic | p-value |
| AUC (ng/ml · hr) | 78.6 (35.7) | 208.7 | 3.6 | <0.01 |
| Cₘₐₓ (ng/ml) | 13.8 (3.2) | 36.7 | 7.3 | <0.01 |
| t_{1/2} (hr) | 3.0 (0.7) | 5.4 | 3.3 | <0.01 |

Pharmacodynamic Statistical Analysis and Consequences of a CES1 Deficiency. When all data including that from the time points 0-1.5 h were included in the dataset, the slow metabolizer was an outlier for the endpoint of MAP (distance: 18.0), while SBP approached statistical significance (distance: 13.3 compared to the critical value 13.8). However, he was not an outlier for the measures of DBP and HR. When the time frame during which MPH absorption generally occurs after oral administration was excluded (i.e. 0-1.5 h) statistical outlier status was achieved for all hemodynamic measures (i.e. SBP, DBP, HR, and MAP). Values prior to dosing as well as maximum values for the slow metabolizer versus the other 19 subjects are shown in Table 2.

TABLE 2
Hemodynamic parameters in the slow metabolizer
versus the 19 study peers prior to dosing with
methylphenidate and the maximum values obtained

| Hemodynamic | Pre-Dose | | Maximum Value | |
|---|---|---|---|---|
| Parameter | Mean (SD) | Outlier | Mean (SD) | Outlier |
| Systolic Blood | 114 (12) | 100 | 120 (10) | 138 |
| Pressure | | | | |
| Diastolic Blood | 65 (9) | 60 | 68 (8) | 78 |
| Pressure | | | | |
| Mean Arterial | 82 (9) | 73 | 85 (8) | 97 |
| Pressure | | | | |
| Pulse | 62 (10) | 70 | 74 (11) | 93 |

Identification of CES1 Polymorphisms. All 14 exons of the CES1 gene were sequenced. Two nonsynonymous, coding region variants of CES1 were identified (FIG. 4). The first was a substitution (p.Gly143Glu) in exon 4 at the second nucleotide (nt) of codon 143 (genomic: nt 9486; cDNA: nt 428), changing G to A (GGG→GAG). This results in the nonconservative amino acid substitution of Glycine 143 to Glutamic acid (Gly143Glu). The second variant identified (p.Asp260fs) was a deletion (T/-) occurring in exon 6 at the last nucleotide (genomic: nt 12754; cDNA: nt 780) of codon 260 (GAT→GA-G). This results in a frameshift (p.Asp260fs) mutation which changes Aspartic acid 260 to Glutamic acid and alters the next 39 residues from the wild-type sequence, before truncating early at a premature stop codon (FIG. 4). Thus, wild-type CES1 and the substitution (p.Gly143Glu) variant are each 567 amino acids long, but the frameshift (p.Asp260fs) variant is limited to 298 amino acids (the first 259 wild-type followed by 39 missense residues and early truncation).

Allelic Frequency of Mutations in Specific Racial and Ethnic Groups. The allelic frequency of these CES1 variants was determined utilizing the described genomic DNA data set. Of those, 455 Caucasians, 117 Black subjects, and 299 Hispanic individuals were studied, and a total of 34, 10, and 12 were identified as heterozygous for p.Gly143Glu, respectively. Thus the minor allele frequency (MAF) of p.Gly143Glu is estimated to be 3.7%, 4.3%, and 2.0% in Caucasian, Black, and Hispanic populations, respectively. There was no deviation from Hardy-Weinberg Equilibrium in any of the populations where the variant was detected. Additionally, p.Gly143Glu was not identified in 54 Asian subjects studied indicating the variant should be considered as rare in Asian population (Table 3). The p.Asp260fs appears to be a rare mutation being found in none of the 925 subjects genotyped with a frequency substantially lower than 1%. There were no statistically significant differences between sexes in the MAF of p.Gly143Glu in any racial or ethnic group. Lastly, genotyping of each of the aberrant metabolizer's biological parents revealed that the father was heterozygous for p.Gly143Glu while the mother was heterozygous for p.Asp260fs. This indicates that the two variants are not in linkage disequilibrium and are thus found on separate alleles.

TABLE 3
Minor allele frequencies (MAF) of CES1 SNP p.Gly143Glu

| | Caucasian | Black | Hispanic | Asian | Total |
|---|---|---|---|---|---|
| GA | n = 34 | n = 10 | n = 12 | n = 0 | n = 56 |
| | (7.5%) | (8.5%) | (4.0%) | (0.0%) | (6.0%) |
| GG | n = 421 | n = 107 | n = 287 | n = 54 | n = 869 |
| | (92.5%) | (91.5%) | (96.0%) | (100.0%) | (94.0%) |
| MAF | C = 3.7% | C = 4.3% | C = 2.0% | C = 0% | C = 3.0% |
| Totals | 455 | 117 | 299 | 54 | 925 |

* The 95% confidence interval of average MAF from four tested populations is −0.57%~5.57%.

Enzymatic activity of CES1 and its mutants on MPH and PNPA hydrolysis. Kinetic parameters (Vₘₐₓ and Kₘ) of CES1 and its mutations for PNPA and d- and l-MPH were determined by measuring the rate of enzymatic production of PNP and RA at different substrate concentrations and fitting the data to the Michaelis-Menten equation using nonlinear regression analysis.

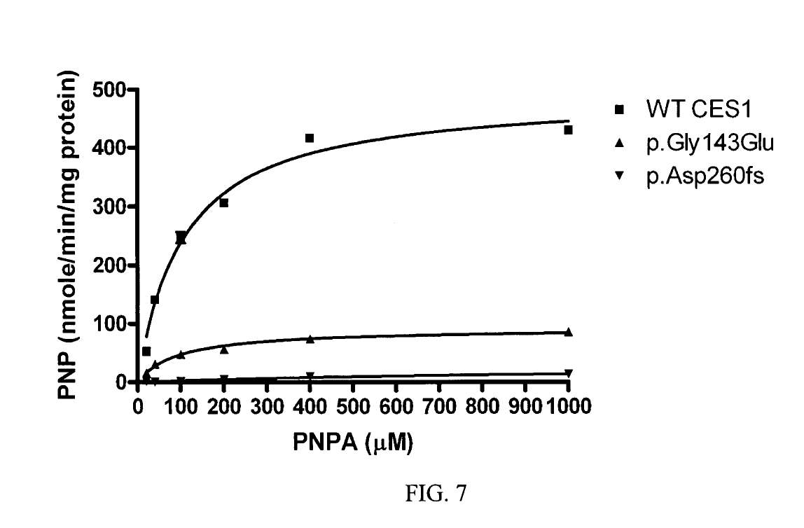

PNPA is a sensitive and established model substrate of CES1 as well as other human esterases. The results demonstrated that WT CES1 exhibited significant catalytic activity on PNPA hydrolysis with Vₘₐₓ and Kₘ values of 493.9 nmole/min/mg protein and 106.6 μM, respectively. Significantly decreased enzymatic activity of p.Gly143Glu and p.Asp260fs were observed. The Vₘₐₓ values of p.Gly143Glu and p.Asp260fs were only 18.6% and 5.7% of that of WT CES1, respectively. The Kₘ value for p.Gly143Glu is comparable to that of WT while p.Asp260fs is approximately 9.5 times greater than that of WT indicating the affinity of p.Asp260fs (but not p.Gly143Glu) to its recognized substrate was noted to be significantly decreased (FIG. 7, Table 4).

TABLE 4
Kinetic parameters of enzymic hydrolysis
of PNPA, l-MPH, and d-MPH.

| Substrates | Enzymes | Vₘₐₓ | Kₘ | Vₘₐₓ/Kₘ |
|---|---|---|---|---|
| PNPA | WT | 493.9 ± 14.3 | 106.6 ± 10.1 | 4.63 |
| | p.Gly143Glu | 92.0 ± 2.9 | 93.2 ± 9.8 | 0.99 |
| | p.Asp260fs | 28.0 ± 6.1 | 950.4 ± 352.1 | 0.03 |
| l-MPH | WT | 1701.0 ± 196.5 | 775.7 ± 161.9 | 2.19 |
| | p.Gly143Glu | N.D. | N.D. | N.D. |
| | p.Asp260fs | N.D. | N.D. | N.D. |
| d-MPH | WT | 177.2 ± 29.6 | 663.5 ± 210.0 | 0.27 |
| | p.Gly143Glu | N.D. | N.D. | N.D. |
| | p.Asp260fs | N.D. | N.D. | N.D. |

Values represent the mean ± S.D. (n = 3).
N.D., not detectable.
Kₘ values are in micromolar.
Vₘₐₓ values for PNPA and MPH are in nmole/min/mg protein and pmole/min/mg protein, respectively.
Vₘₐₓ/Kₘ values for PNPA and MPH are in ml/min/mg protein and μl/min/mg protein, respectively.

The MPH hydrolysis study demonstrated that WT CES1 exhibited substantial stereoselectivity relative to catalytic efficiency with l-MPH favored over the d-isomer with a Vₘₐₓ of 1701.0 and 177.2 pmole/min/mg protein, respectively. These stereoselective actions were entirely consistent with the inventor's previous clinical observations and those of other investigators. The catalytic activity produced by CES1 mutants, p.Gly143Glu and p.Asp260fs towards MPH hydrolysis were too low to be determined under the inventor's experimental conditions even when a very high S9 concentration (2 mg/ml) was employed. (FIG. 6, Table 4).

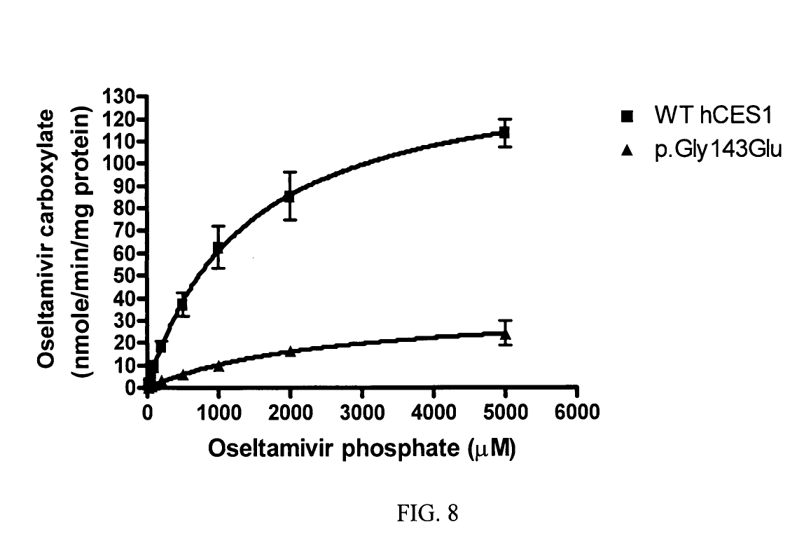

This invention confirms that hCES1 is the major hydrolase governing the activation of the prodrug oseltamivir via cleavage of the ester group and that OP serves as an excellent substrate of hCES1. It demonstrates that OP is rapidly metabolized to its active form OC with the Vₘₐₓ and Kₘ values of 145.3 nmole/min/mg protein and 1381.6 μM, respectively. The hCES1 variants p.Gly143Glu and p.Asp260fs displayed substantially decreased enzymatic activity towards oseltamivir hydrolysis. The Vmax value of p.Gly143Glu was determined to be 37.1 nmole/min/mg protein, which is approximately one fourth of that of WT hCES1. Similar to the inventor's experimental observations with MPH, the incubation of p.Asp260fs did not result in any detectable hydrolysis of OP as measured by conversion to OC (FIG. 8).

Discussion. The glycine 143 is a crucial residue to hCES1 catalytic function.^{20,24} The inventor's functional studies demonstrated that the majority of functional activity of hCES1 is lost in p.Gly143Glu after the glycine 143 is substituted with glutamic acid. Furthermore, although apparently a rare mutation in the general population, the premature enzyme p.Asp260fs resulting from a frame shift mutation could be viewed as a totally dysfunctional CES1 mutant. The inventor's data strongly suggest that the individuals who carry either of those SNPs are expected to display abnormal PK and therapeutic response to the drugs metabolized (deactivated or activated) by hCES1.

This study identified two variants in the CES1 gene. One in exon 4 is located in codon 143 (GGG→GAG) and leads to the nonconservative Glycine 143 to Glutamic acid amino acid substitution (p.Gly143Glu). A deletion in exon 6 at codon 260 results in a frameshift mutation that alters residues 260-299 before truncating early at a premature stop codon (p.Asp260fs). These mutations led to grossly elevated total MPH blood concentrations along with a distortion in the typical isomer disposition within an individual who was heterozygous for both mutations on separate alleles of the CES1 gene.

In order to determine if the variations in CES1 activity led to a quantifiable difference in the pharmacodynamic effects of MPH, the inventor compared the hemodynamic response of this individual to his study peers. The subject was an outlier for all endpoints (SBP, DBP, HR, and MAP) using data points obtained 1.5 hours after MPH administration. Importantly, therapeutic doses of MPH can occasionally cause adverse cardiovascular effects. In rare instances stroke or sudden death has been reported in patients with underlying risk factors (FDA, FDA Directs ADHD Drug Manufacturers to Notify Patients about Cardiovascular Adverse Events and Psychiatric Adverse Events). The present results suggest a potential for an increased risk of adverse events in individuals with either identified CES1 variants, a risk which would be expected to increase with the thrice daily dosing typically recommended with immediate release MPH, or the use of the once daily formulations which presently dominate the market.

To reveal the molecular genetic basis of the profoundly altered pharmacokinetics and pharmacodynamic effects of MPH in the deficient metabolizer, the catalytic function of each CES1 variant was investigated utilizing the cells stably expressing WT CES1, p.Gly143Glu, and p.Asp260fs. The results demonstrated that the catalytic efficiency of CES1 was dramatically decreased in p.Gly143Glu and p.Asp260fs. This observation is in good agreement with the hypothesis of CES1 mediated catalysis mechanism recently proposed. The CES1 enzyme belongs to a larger family of serine hydrolases, which include human acetylcholinesterase (AcChE) and butyrylcholine esterase (BuChE). Crystal structures of CES1, AcChE, and BuChE indicate that each has an analogous active site groove containing a catalytic triad consisting of a serine, a glutamic acid, and a histidine residue. For CES1 the corresponding active site triad residues are serine 221 (S), glutamic acid 354 (E), and histidine 468 (H; shown in bold in FIG. 4). A series of three consecutive glycine residues is also located in the active site of CES1 (Gly141-143) and create what is now referred to as an oxyanion hole. The oxyanion hole is thought to stabilize substrate-enzyme intermediates and thus, would prove fundamental to CES1 functionality. The catalytic triad and oxyanion hole are evolutionarily conserved both across species and within related serine hydrolases. In p.Asp260fs the subsequent frameshift and early truncation as a result of deletion of nucleotide 780 results in a protein missing two of the three conserved catalytic triad residues (FIG. 4). However, the apparent rarity of the p.Asp260fs indicates this mutation is of considerably less clinical concern than p.Gly143Glu. When the glycine in BuChE (analogous to Gly143 in CES1) was mutated, both substrate affinity and catalysis were markedly reduced or abolished. This suggests that the mutation resulting in substitution of Gly143 with glutamic acid (i.e. p.Gly143Glu) is of critical importance and could likely result in dysfunctional CES1. Taken together, these findings support the hypothesis that both identified CES1 variants are likely to result in significant loss of CES1 activity, which had been demonstrated by these in vitro functional studies, and either is sufficient to significantly disrupt catalytic activity. The aberrant metabolizer was heterozygous for both mutations and each mutation occurred on a different allele. Due to the estimated frequencies of each variants, having one of these mutants on both alleles is expected to be an extremely rare occurrence. Nevertheless, the serendipitous identification of the subject's phenotype was key to discovering and exploring the two variants described.

The CES1 enzyme catalyzes the hydrolysis of drugs from numerous classes.

Ester cleavage generally produces inactive metabolite(s) as in the case of MPH. However, CES1 is also known to be involved in the generation of active metabolites including the conversion of heroin to monoacetylmorphine and morphine. Of perhaps greater significance is the role of CES1 in the activation of prodrugs including a number of angiotensin converting enzyme (ACE) inhibitors (e.g., quinapril) and the anti-influenza agent oseltamivir. There is at least one research report in which a SNP in the promoter region of CES1, A(−816)C, favorably influenced the antihypertensive response to the prodrug Angiotensin-Converting Enzyme inhibitor imidapril which highlights the importance of adequate and functional CES1 when administering prodrug substrates of this enzyme. Additionally, functional CES1 is required for the mediation of transesterification reactions. With regard to drugs of abuse, the existence of an unrecognized CES1 deficiency could potentially lead to toxicities and/or fatal exposures misinterpreted as intentional or accidental drug overdoses if the judgment were made on the basis of antemortem or postmortem concentrations. Furthermore, these hydrolytic reactions can proceed on a stereoselective basis resulting in a distortion of the anticipated disposition of a racemic compound such as dl-MPH (FIG. 3). The effect of each individual variant on the metabolism of other substrates will need to be further investigated. Finally, the presence of dysfunctional or non-functional CES1 may result in a poor response to a variety of prodrugs formulated as esters for the purpose of enhanced bioavailability due to the inability of CES1 to cleave and liberate the active therapeutic moiety.

Further studies are needed to define the clinical significance of each allele as it relates to drug response and adverse events. Beyond MPH, these variants in the CES1 gene are pertinent to the use of a diverse group of therapeutic agents as well as individuals abusing illicit substances. Genotyping individuals participating in well designed clinical studies of known CES1 substrates will be an initial step in evaluating the true clinical significance of these variants and their contribution to individualized pharmacotherapy.

All of the methods and compositions disclosed and claimed herein can be made and executed without undue experimentation in light of the present disclosure. While the compositions and methods of this invention have been described in terms of preferred embodiments, it will be apparent to those of skill in the art that variations may be applied to the methods and compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Laizure S C, Mandrell T, Gades N M, et al. Cocaethylene metabolism and interaction with cocaine and ethanol: role of carboxylesterases. Drug Metab Dispos 2003; 31: 16-20.
2. Redinbo M R, Bencharit S, Potter P M. Human carboxylesterase 1: from drug metabolism to drug discovery. Biochem Soc Trans 2003; 31: 620-4.
3. Biederman J. Attention-deficit/hyperactivity disorder: a selective overview. Biol Psychiatry 2005; 57: 1215-20.
4. Faraone S V, Sergeant J, Gillberg C, et al. The worldwide prevalence of ADHD: is it an American condition? World Psychiatry 2003; 2: 104-113.
5. Markowitz J S, Straughn A B, Patrick K S. Advances in the pharmacotherapy of attention-deficit-hyperactivity disorder: focus on methylphenidate formulations. Pharmacotherapy 2003; 23: 1281-99.
6. Patrick K S, Caldwell R W, Ferris R M, et al. Pharmacology of the enantiomers of threo-methylphenidate. J Pharmacol Exp Ther 1987; 241: 152-8.
7. Patrick K S, Kilts C D, Breese G R. Synthesis and pharmacology of hydroxylated metabolites of methylphenidate. J Med Chem 1981; 24: 1237-40.
8. Sun Z, Murry D J, Sanghani S P, et al. Methylphenidate is stereoselectively hydrolyzed by human carboxylesterase CES1A1. J Pharmacol Exp Ther 2004; 310: 469-76.
9. Patrick K S, Gonzalez M A, Straughn A B, et al. New methylphenidate formulations for the treatment of attention-deficit/hyperactivity disorder. Expert Opin Drug Deliv 2005; 2: 121-43.
10. Patrick K S, Straughn A B, Minhinnett R R, et al. Influence of ethanol and gender on methylphenidate pharmacokinetics and pharmacodynamics. Clin Pharmacol Ther 2007; 81: 346-53.
11. Imai T. Human Carboxylesterase Isozymes: Catalytic Properties and Rational Drug Design. Drug Metabolism and Pharmacokinetics 2006; 21: 173-185.
12. Rosner B. Fundamentals of Biostatistics, Fifth Edition. Duxbury Press, Pacific Grove, Calif. 2000;
13. Penny K. Appropriate critical values when testing for a single multivariate outlier by using the Mahalanobis distance. Applied Statistics 1996; 45: 73-81.
14. Kelly R P, Yeo K P, Teng C-H, et al. Hemodynamic Effects of Acute Administration of Atomoxetine and Methylphenidate. J Clin Pharmacol 2005; 45: 851-855.
15. Findling R, Short E, Manos M. Short-Term Cardiovascular Effects of Methylphenidate and Adderall. Journal of the American Academy of Child and Adolescent Psychiatry 2001; 40: 525-529.
16. Volkow N D, Wang G-J, Fowler J S, et al. Cardiovascular effects of methylphenidate in humans are associated with increases of dopamine in brain and of epinephrine in plasma. Psychopharmacology 2003; 166: 264-270.
17. FDA Directs ADHD Drug Manufacturers to Notify Patients about Cardiovascular Adverse Events and Psychiatric Adverse Events (www.ncbi.nlm.nih.gov/entrez/query.fcgi?cmd=Retrieve&db=pubmed&dopt=Abstract&list_uids=12589522).
18. Satoh T, Taylor P, Bosron W F, et al. Current Progress on Esterases: From Molecular Structure to Function. Drug Metab Dispos 2002; 30: 488-493.
19. Fleming C D, Bencharit S, Edwards C C, et al. Structural Insights into Drug Processing by Human Carboxylesterase 1: Tamoxifen, Mevastatin, and Inhibition by Benzil. Journal of Molecular Biology 2005; 352: 165-177.
20. Masson P, Froment M-T, Gillon E, et al. Hydrolysis of oxo- and thio-esters by human butyrylcholinesterase. Biochimica et Biophysica Acta (BBA)—Proteins & Proteomics 2007; 1774: 16-34.
21. Markowitz, J. S. and K. S. Patrick (2008). “Differential pharmacokinetics and pharmacodynamics of methylphenidate enantiomers: Does chirality matter?” J Clin Psychopharmacol (In Press).
22. Shi D, Yang J, Yang D, LeCluyse E L, Black C, You L, et al. Anti-influenza prodrug oseltamivir is activated by carboxylesterase human carboxylesterase 1, and the activation is inhibited by antiplatelet agent clopidogrel. J Pharmacol Exp Ther 2006 December; 319(3):1477-84.
23. www.fda.gov/cder/foi/nda/99/21087_Tamiflu_pharmr_P1.pdf accessed Dec. 12, 2007.
24. Tang M, Mukundan M, Yang J, Charpentier N, LeCluyse E L, Black C, et al. Antiplatelet agents aspirin and clopidogrel are hydrolyzed by distinct carboxylesterases, and clopidogrel is transesterificated in the presence of ethyl alcohol. J Pharmacol Exp Ther 2006 December; 319(3):1467-76.
25. Liederer B M, Borchardt R T. Enzymes involved in the bioconversion of ester-based prodrugs. J Pharm Sci 2006 June; 95(6):1177-95.
26. Li B, Sedlacek M, Manoharan I, Boopathy R, Duysen E G, Masson P, et al. Butyrylcholinesterase, paraoxonase, and albumin esterase, but not carboxylesterase, are present in human plasma. Biochem Pharmacol 2005 Nov. 25; 70(11):1673-84.
27. Wu K M, Farrelly J G. Regulatory perspectives of Type II prodrug development and time-dependent toxicity management: nonclinical Pharm/Tox analysis and the role of comparative toxicology. Toxicology 2007 Jul. 1; 236(1-2):1-6.
Guilfoyle et al., Nucleic Acids Research, 25:1854-1858, 1997.
Walker et al., Proc. Natl. Acad. Sci. USA, 89:392-396 1992.
Kwoh et al., Proc. Natl. Acad. Sci. USA, 86: 1173, 1989.
Frohman, In: PCR Protocols: A Guide To Methods And Applications, Academic Press, N.Y., 1990.
Ohara et al., Proc. Natl. Acad. Sci. USA, 86: 5673-5677, 1989.
Gobom et al., Anal. Chem., 72(14):3320-3326, 2000.
Bucknall et al., J. Am. Soc. Mass Spectrom., 13(9):1015-1027, 2002.
Gobom et al., Anal. Chem., 72(14):3320-3326, 2000.

1. A method of diagnosing reduced carboxylesterase-1 function in a subject, wherein the method comprises detecting the presence or absence of at least one of 12754T>del or Gly143Glu (9486G>A) in the carboxylesterase-1 gene in a biological sample from the subject, wherein the presence of at least one of 12754T>del or Gly143Glu (9486G>A) indicates that the subject has reduced carboxylesterase-1 function.

2-3. (canceled)

4. The method of claim 1, wherein the method comprises detecting the presence or absence of both of 12754T>del or Gly143Glu (9486G>A) in the carboxylesterase-1 gene.

5. The method of claim 1, wherein the detecting comprises real-time PCR (rtPCR).

6-12. (canceled)

13. The method of claim 1, wherein the subject is a human.

14. The method of claim 1, wherein the biological sample is blood, sputum, saliva, mucosal scraping, or a tissue biopsy.

15. (canceled)

16. The method of claim 1, further comprising making a decision on the therapy for the subject.

17. (canceled)

18. The method of claim 16, wherein said decision comprises determining an appropriate dose of a drug selected from an opioid, meperidine, a dopaminergic or noradrenergic drug, methylphenidate, an ACE inhibitor, quinapril, enalapril, benzapril, imidapril, delapril, pemocapril, cilazapril, an anesthetic, lidocaine, lovastatin, an antiviral drug, oseltamivir, an anti-cancer drug, or irinotecan.

19. The method of claim 1, wherein the method is further defined as a method of determining the sensitivity of the subject to a compound.

20. The method of claim 19, wherein the compound is selected from the group consisting of heroin, cocaine, a toxin, a chemical warfare agent, sarin nerve gas, soman, tabun, an insecticide, and an organophosphate insecticide.

21. The method of claim 1, wherein DNA or RNA is isolated from the sample.

22. (canceled)

23. The method of claim 1, wherein a nucleic acid probe is hybridized to DNA obtained or derived from the sample.

24. The method of claim 23, wherein the probe is detectably labeled.

25-29. (canceled)

30. The method of claim 1, wherein at least part of the carboxylesterase-1 gene of the subject is amplified prior to detection.

31. (canceled)

32. The method of claim 1, wherein the subject is heterozygous for at least one of said 12754T>del or Gly143Glu (9486G>A).

33. The method of claim 1, wherein the subject is homozygous for at least one of said 12754T>del or Gly143Glu (9486G>A).

34. The method of claim 1, wherein the subject has neither said 12754T>del or Gly143Glu (9486G>A).

35. The method of claim 1, wherein the detecting comprises sequencing at least part of the carboxylesterase-1 gene of the subject.

36. A kit for detecting: (i) the presence or absence of Gly143Glu (9486G>A) in a carboxylesterase-1 gene comprising a nucleic acid probe in a suitable container means, wherein the nucleic acid probe can selectively bind the 9486 nucleotide of the carboxylesterase-1 gene; or the presence or absence of 12754T>del in a carboxylesterase-1 gene comprising a nucleic acid probe in a suitable container means, wherein the nucleic acid probe can selectively bind the 12754 nucleotide of the carboxylesterase-1 gene; or (iii) both nucleic acid probes of (i) and (ii).

37-50. (canceled)

51. A method of assessing the effect of carboxylesterase-1 (CES1) activity on a candidate substance comprising:
(a) providing a cell that expresses mutant CES1 or overexpresses, relative to a normal cell, wild-type CES1;
(b) contacting said cell with said candidate substance; and
(c) assessing the effect of CES1 on said candidate substance, the effect of said candidate substance on CES1 expression or activity, or the effect of said candidate substance on said cell.

52-70. (canceled)
